(19) United States
(12) Patent Application Publication
(10) Pub. No.: US 2017/0232439 A1
(43) Pub. Date: Aug. 17, 2017

(54) SEPARATION OF LOW-ABUNDANCE CELLS FROM FLUID USING SURFACE ACOUSTIC WAVES

(71) Applicants: Carnegie Mellon University, Pittsburgh, PA (US); Massachusetts Institute of Technology, Cambridge, MA (US); The Penn State Research Foundation, University Park, PA (US)

(72) Inventors: Subra Suresh, Pittsburgh, PA (US); Peng Li, State College, PA (US); Ming Dao, West Roxbury, MA (US); Yuchao Chen, State College, PA (US); Xiaoyun Ding, State College, PA (US); Tony Jun Huang, State College, PA (US); Zhangli Peng, Mishawaka, IN (US)

(73) Assignees: Carnegie Mellon University, Pittsburgh, PA (US); Massachusetts Institute of Technology, Cambridge, MA (US); The Penn State Research Foundation, University Park, PA (US)

(21) Appl. No.: 15/503,182

(22) PCT Filed: Aug. 11, 2015
(86) PCT No.: PCT/US2015/044712

§ 371 Date: Feb. 10, 2017

Related U.S. Application Data
(60) Provisional application No. 62/035,926, filed on Aug. 11, 2014.
(60) Provisional application No. 62/129,472, filed on Mar. 6, 2015.

(51) Int. Cl. B01L 3/00; C12Q 1/24

(52) U.S. Cl. CPC B01L 3/502761; C12Q 1/24; B01L 2400/0436; B01L 2300/0864; B01L 2300/0816; B01L 2200/0652

(57) ABSTRACT

An apparatus for sorting cells from a mixed population of cells using surface acoustic waves is described. Methods for separating cancer cells from a mixed population of cells are provided. Methods for separating cells or particles having different size, density and/or compressibility properties are also provided.

### RELATED APPLICATIONS

The present application claims priority under 35 U.S.C. §119(e) to U.S. Provisional Application No. 62/035,926, filed Aug. 11, 2014 and to U.S. Provisional Application No. 62/129,472, filed Mar. 6, 2015 each of which is incorporated herein by reference.

### FEDERALLY SPONSORED RESEARCH

This invention was made with government support under 1DP2OD007209-01 awarded by the National Institutes of Health. The government has certain rights in the invention.

### BACKGROUND OF THE INVENTION

Many applications in biology and medicine call for efficient and reliable separation of particles and cells for disease diagnosis, genetic analysis, drug screening, and therapeutics (1-6). Cells can be separated on the basis of their surface molecular markers or physical characteristics such as density, size, stiffness, or electric impedance (7-10). When separating cells with distinct physical properties, using methods that exploit differences in cells' physical parameters could be advantageous due to their label-free nature and ease of use (11, 12). Many techniques are available to separate cells based on physical properties; they include filtration, centrifugation, acoustics, optics, and dielectrophoresis (13-27). The so-called “acoustic tweezers” technologies, which can perform highly precise cell manipulations, are particularly promising for cell-separation applications and offer additional advantages in ease of use and versatility (27, 29). Despite these advantages, acoustic separation has not been widely used in practical cell-separation applications due to their relatively low separation sensitivity and efficiency.

### SUMMARY OF THE INVENTION

Aspects of the disclosure relate to a method for separating cancer cells from a mixed population of cells, comprising flowing a sample containing a mixed population of cells through a channel, wherein the mixed population of cells includes cancer cells and non-cancer cells, subjecting the sample to a surface acoustic wave (SAW), causing the sample to separate into two flowing streams of sample, wherein the first flowing stream of sample has cancer cells and the second flowing stream has non-cancer cells, wherein the cancer cells comprise less than 10% of the total mixed population of cells. Other aspects of the disclosure relate to a method for separating cancer cells from a mixed population of cells, comprising flowing a sample containing a mixed population of cells through a channel, wherein the mixed population of cells includes cancer cells, wherein the cancer cells are not cultured cancer cells, and non-cancer cells, subjecting the sample to a surface acoustic wave (SAW), causing the sample to separate into two flowing streams of sample, wherein the first flowing stream of sample has cancer cells and the second flowing stream has non-cancer cells. In some embodiments, the cancer cells comprise less than 10% of the total mixed population of cells. In some embodiments, the cancer cells comprise less than 5% of the total mixed population of cells. In some embodiments, the cancer cells comprise less than 2% of the total mixed population of cells. In some embodiments, the cancer cells comprise less than 1% of the total mixed population of cells. In some embodiments, the channel comprises a compartment having a region that connects with multiple compartments. In some embodiments, the cancer cells are circulating tumor cells (CTCs). In some embodiments, the CTCs comprise 1-100 cancer cells in one mL of blood and optionally, wherein the CTCs comprise 1-10 cancer cells in 7.5 mL of blood. In some embodiments, the CTCs comprise 60-1000 cells in one mL of blood. In some embodiments, the cancer cells separated from the mixed cell population maintain high cell viability and integrity. In some embodiments, greater than 80% separation efficiency of the cancer cells from the mixed cell population is achieved. In some embodiments, greater than 85% separation efficiency of the cancer cells from the mixed cell population is achieved. In some embodiments, greater than 90% separation efficiency of the cancer cells from the mixed cell population is achieved. In some embodiments, greater than 95% separation efficiency of the cancer cells from the mixed cell population is achieved. In some embodiments, at least 1 mL of sample per hour is processed and optionally, wherein a flow rate of the fluid is 1-500 μL/min. In some embodiments, the surface acoustic wave is generated by at least two interdigital transducers (IDT). In some embodiments, the surface acoustic wave is generated by at least two segmented interdigital transducers (S-IDT). In some embodiments, the number of segments of the segmented interdigital transducer range from 5 to 30. In some embodiments, the length of any of the segments range from 100 μm to 1000 μm. In some embodiments, the segmented interdigital transducer has 15 segments, wherein the length of the segments is 250 μm.

Other aspects of the disclosure relate to a method for separating label-free circulating tumor cells (CTCs) from a mixed population of cells in a non-invasive manner, comprising subjecting a fluid biological sample containing the CTCs to a surface acoustic wave (SAW) to separate the CTCs from other cells in the biological sample. In some embodiments, the method is for monitoring the disease progress of a patient, wherein the fluid biological sample is isolated from a patient and the progress of the disease in the patient is based on the separated CTCs. In some embodiments, the method is for determining genetic mutations of a cancer patient, wherein the fluid biological sample is isolated from a cancer patient and mutations present in the CTCs are determined. In some embodiments, the method is for predicting the therapy outcome of a patient, wherein the fluid biological sample is isolated from the patient and the therapy outcome of the patient is determined based on the separated CTCs. In some embodiments, the method is for diagnosing cancer, wherein the fluid biological sample is isolated from the patient, and the cancer is diagnosed based on the separated CTCs. In some embodiments, the cancer is metastatic cancer. In some embodiments, the biological sample is a blood sample. In some embodiments, the methods further comprise isolating the CTCs. In some embodiments, the methods further comprise using any apparatus disclosed herein. In some embodiments, the surface acoustic wave direction is at a non-oblique angle to the direction of flow in the channel. In some embodiments, the surface acoustic wave direction is at an oblique angle to the direction of flow in the channel. In some embodiments, the surface acoustic wave direction is at an angle ranging from 0-10 degrees to the direction of flow in the channel. In some embodiments, the surface acoustic wave direction is at an angle ranging from 10-15 degrees to the direction of flow in the channel. In some embodiments, the surface acoustic wave direction is at an angle ranging from 15-30 degrees to direction of flow in the channel. In some embodiments, the surface acoustic wave direction is at an angle ranging from 30-45 degrees to the direction of flow in the channel. In some embodiments, the surface acoustic wave direction is at a 30 degree angle to the direction of flow in the channel. In some embodiments, the SAW has an acoustic power ranging from 19 to 31 dBm. In some embodiments, the SAW has an acoustic power of 19, 23, 27, or 31 dBm.

Other aspects of the disclosure relate to an apparatus for sorting cells from a mixed population of cells, comprising a surface, a channel on the surface, the channel having an inlet end and an outlet end, wherein the inlet end comprises at least one inlet and the outlet end comprises at least two outlets, the channel having a direction from the inlet end to the outlet end; a first surface acoustic wave (SAW) generator, wherein the SAW generator is a segmented interdigital transducer (S-IDT), a second SAW generator, wherein the second SAW generator is a S-IDT, the first and second SAW generators being operably configured on the surface, and on opposing sides of the channel to generate a SAW within the channel between the inlet end and outlet end of the channel and having a SAW direction. In some embodiments, the SAW direction is disposed at a 10-45 degree angle to the channel direction. In some embodiments, the number of segments of the segmented interdigital transducer range from 5 to 30. In some embodiments, the length of any of the segments range from 100 μm to 1000 μm. In some embodiments, the segmented interdigital transducer has 15 segments, wherein the length of the segments is 250 μm.

Other aspects of the disclosure relate to an apparatus for sorting cells from a mixed population of cells, comprising a surface, a channel on the surface, the channel having an inlet end and an outlet end, wherein the inlet end comprises at least one inlet and the outlet end comprises at least two outlets the channel having a direction from the inlet end to the outlet end, wherein the surface acoustic wave direction is disposed at an angle to the channel direction, and first and second SAW generators operably configured on the surface, and on opposing sides of the channel to generate a SAW within the channel between the inlet end and outlet end of the channel and having a SAW direction, wherein the SAW direction is disposed at a 10-25 degree angle. In some embodiments, the SAW direction is disposed at a 15 degree angle. In some embodiments, the surface is a piezoelectric substrate; and the first and second SAW generators each comprise electrodes supported by the surface. In some embodiments, the apparatus is a microfluidic device; the channel is a microchannel; and the microchannel has at least one cross-sectional dimension less than 1 mm. In some embodiments, the SAW direction is at a non-oblique angle to the channel direction. In some embodiments, the SAW direction is at an oblique angle to the channel direction. In some embodiments, the SAW direction is at an angle ranging from 0-10 degrees to the channel direction. In some embodiments, the SAW direction is at an angle ranging from 10-15 degrees to the channel direction. In some embodiments, the SAW direction is at an angle ranging from 15-30 degrees to the channel direction. In some embodiments, the SAW direction is at an angle ranging from 30-45 degrees to the channel direction. In some embodiments, the SAW direction is at a 30 degree angle to the channel direction. In some embodiments, the surface forms a wall of the channel. In some embodiments, the first and second SAW generators are configured to emit an acoustic output ranging from 17-23 dBm (e.g., 50 to 200 mW). In some embodiments, the first and second SAW generators emit an acoustic power ranging from 19 to 31 dBm. In some embodiments, the first and second SAW generators emit an acoustic power of about 19, 23, 27, or 31 dBm.

Other aspects of the disclosure relate to a method for separating cells or particles based on a cellular or particle property from a mixed population of cells, comprising flowing a sample containing a mixed population of cells or particles through a channel, wherein the mixed population of cells or particles includes a first population of cells or particles having a first value for the property, and a second population of cells or particles having a second value for the property, subjecting the sample to a surface acoustic wave (SAW), causing the sample to separate into two flowing streams of sample, wherein the first flowing stream of sample has the first population of cells or particles and the second flowing stream has the second population of cells or particles, wherein the first population of cells or particles and the second population of cells or particles have a similar size. In some embodiments, the property is compressibility. In some embodiments, the first value for compressibility and the second value for compressibility are non-identical. In some embodiments, the first value for compressibility and the second value for compressibility differ by at least 0.23×10-10 Pa-1. In some embodiments, the first value for compressibility and the second value for compressibility differ by at least 5.5%. In some embodiments, the methods further comprise a third population of cells or particles having a third value for compressibility, wherein the third population of cells or particles separates into a third flowing stream. In some embodiments, the methods further comprise a fourth population of cells or particles having a fourth value for compressibility, wherein the fourth population of cells or particles separates into a fourth flowing stream. In some embodiments, the property is density. In some embodiments, the first value for density and the second value for density are non-identical. In some embodiments, the first value for density and the second value for density differ by at least 49 kg/m3. In some embodiments, the first value for density and the second value for density differ in diameter by at least 5%. In some embodiments, the methods further comprise a third population of cells or particles having a third value for density, wherein the third population of cells or particles separates into a third flowing stream. In some embodiments, the methods further comprise a fourth population of cells having a fourth density, wherein the fourth population of cells or particles separates into a fourth flowing stream. In some embodiments, the separation efficiency of at least one cell population is at least 85%. In some embodiments, the separation efficiency of at least one cell population is at least 90%. In some embodiments, the separation efficiency of at least one cell population is at least 95%. In some embodiments, the separation efficiency of at least one cell population is at least 97%. In some embodiments, the methods further comprise using any apparatus disclosed herein.

Other aspects of the disclosure relate to a method for separating cells or particles of different size from a mixed population of cells or particles, comprising flowing a sample containing a mixed population of cells or particles through a channel, wherein the mixed population of cells or particles includes a first population of cells or particles having a first size, and a second population of cells or particles having a second size, subjecting the sample to a surface acoustic wave (SAW), causing the sample to separate into two flowing streams of sample, wherein the first flowing stream of sample has the first population of cells or particles and the second flowing stream has the second population of cells, wherein the first population of cells and the second population of cells or particles have at least two other properties in common. In some embodiments, the first size and the second size are non-identical. In some embodiments, the first size and the second size differ by at least 2.6 μm in diameter. In some embodiments, the first size and the second size differ in diameter by at least 27%. In some embodiments, the methods further comprise a third population of cells or particles having a third size, wherein the third population of cells or particles separates into a third flowing stream. In some embodiments, the methods further comprise a fourth population of cells having a fourth size, wherein the fourth population of cells or particles separates into a fourth flowing stream. In some embodiments, the separation efficiency of at least one cell population is at least 85%. In some embodiments, the separation efficiency of at least one cell population is at least 90%. In some embodiments, the separation efficiency of at least one cell population is at least 95%. In some embodiments, the separation efficiency of at least one cell population is at least 97%. In some embodiments, the velocity of flow is about 1.5 mm/s. In some embodiments, the angle between the sonic acoustic wave (SAW) and the flow direction is set at about 15 degrees. In some embodiments, the SAW is generated by an interdigital transducer having electrode fingers, wherein the electrode fingers are about 4 mm in length. In some embodiments, the methods further comprise using any apparatus disclosed herein.

Other aspects of the disclosure relate to a method for separating cells or particles from a mixed population of cells or particles in a fluid, comprising identifying at least one measurement of a cell or particle, determining at least one parameter of the method based on at least one measurement of the cell or particle, wherein the method comprises the steps of, flowing a sample containing a mixed population of cells or particles through a channel, wherein the flowing sample has a flow rate, subjecting the sample to a surface acoustic wave (SAW), wherein the SAW is at an angle with respect to the direction of the flow in the channel, causing the sample to separate into at least two flowing streams of cells or particles. In some embodiments, the measurement is a size measurement, a density measurement, or a compressibility measurement. In some embodiments, the size measurement is a volume, or a radius of the cell or particle. In some embodiments, the methods further comprise taking at least one measurement of the fluid and determining at least one parameter of the method based on the measurement of the fluid. In some embodiments, the measurement of the fluid is the density of the fluid, the compressibility of the fluid, or the viscosity of the fluid. In some embodiments, at least two measurements of the cell or particle are taken, and wherein at least one parameter of the method is based on at least two measurements of the cell or particle. In some embodiments, at least three measurements of the cell or particle are taken, and wherein at least one parameter of the method is based on at least three measurements of the cell or particle. In some embodiments, the methods further comprise taking at least two measurement of the fluid and determining at least one parameter of the method based on at least two measurements of the fluid. In some embodiments, the methods further comprise taking at least three measurement of the fluid and determining at least one parameter of the method based on at least three measurements of the fluid. In some embodiments, the parameter is the angle of the surface acoustic wave to the direction of flow in the channel. In some embodiments, the parameter is the acoustic power of the SAW generators. In some embodiments, the parameter is the flow rate. In some embodiments, at least two parameters of the method are determined. In some embodiments, at least three parameters of the method are determined. In some embodiments, greater than 80% separation efficiency of the cells or particles from the mixed population of cells or particles is achieved. In some embodiments, greater than 85% separation efficiency of the cells or particles from the mixed population of cells or particles is achieved. In some embodiments, greater than 90% separation efficiency of the cells or particles from the mixed population of cells or particles is achieved. In some embodiments, greater than 95% separation efficiency of the cells or particles from the mixed population of cells or particles is achieved. In some embodiments, the cells separated from the mixed population of cells maintain high cell viability and integrity. In some embodiments, the surface acoustic wave is generated by at least two interdigital transducers (IDT). In some embodiments, the surface acoustic wave is generated by at least two segmented interdigital transducers (S-IDT). In some embodiments, the number of segments of the segmented interdigital transducer range from 5 to 30. In some embodiments, the length of any of the segments range from 100 μm to 1000 μm. In some embodiments, the segmented interdigital transducer has 15 segments, wherein the length of the segments is 250 μm.

Other aspects of the disclosure relate to a method for preparing a device for separating cells or particles from a mixed population of cells or particles in a fluid, comprising determining magnitude of an acoustic radiation force acting on a particle, wherein the magnitude of the acoustic radiation force acting on the particle is a function of the volume, density, and/or compressibility of the particle, and the power of RF signal applied to the device, wherein the magnitude of acoustic radiation force is indicative of an optimal angle in a channel for separating cells or particles from the mixed population of cells or particles in the fluid, and setting the device to include the optimal angle in order to separate the cells or particles. In some embodiments, the methods further comprise identifying a drag force of a cell or particle, wherein the drag force is expressed as: F_{d}=−6πμRₚuᵣ and wherein the acoustic radiation force is expressed as:

$${F}_{r} = - ( \frac{π {p}_{0}^{2} {V}_{p} {β}_{w}}{2 λ} ) φ ( β , ρ ) sin ( 2 ky ) ,$$

where p₀, λ, Vₚ, ρₘ, ρₚ, βₘ, βₚ, μ, Rₚ, and uᵣ are the acoustic pressure, acoustic wavelength, volume of the particle, density of the medium, density of the particle, compressibility of the medium, compressibility of the particle, viscosity of the medium, radius of the particle, and relative velocity of the particle, respectively, y is the coordinate as shown in FIG. 1, Panel B and k is the wavenumber of the standing acoustic wave.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, claims, and figures. Other features, objects, and advantages of the invention will be apparent from the description and from the claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 Panel A shows a schematic illustration of the working principle and exemplary device structure. The drawing depicts a tilted-angle standing surface acoustic wave (taSSAW)-based cell separation device. Panel B is a schematic showing the separation process for 10 μm diameter polystyrene beads in the taSSAW working region and the outlet region. Panel C is a schematic showing 2 μm diameter polystyrene beads in the taSSAW working region and the outlet region.

FIG. 2 Panel A shows continuous separation of fluorescent polystyrene beads of 7.3 μm (arrow labeled green) and 9.9 μm (arrow labeled red) diameter. Stacked images along the flow direction of the microchannel showing the separation process. Pressure nodal lines are super-posed as dashed lines for illustration purpose. Pane B shows a schematic of the device indicating the portion (boxed in) where the enlarged drawing shown in Panel A is taken from. Panel C shows separation efficiency is analyzed by measuring the distribution of the fluorescence intensity. The fluorescence profile represents the lateral position of the beads after their egress from the working region.

FIG. 3 Is a simulation of cell separation based on compressibility. Computed trajectories of leukemia cells (assuming a softer compressibility of 3.75×10⁻¹⁰ Pa⁻¹) versus normal leukocytes (with a normal compressibility of 3.99×10⁻¹⁰ Pa⁻¹), with the same cell size (12 μm).

FIG. 4 Panel A shows a numerical simulation and experimental demonstration of a particle-separation processes. Optical images showing the separation of beads and cells from experiments; the super-posed areas are simulation results for the same parameters. Trajectory comparison between simulations and experiments are explored for different bead sizes. Panel B shows a trajectory comparison between simulations and experiments that are performed with different input powers. Panel C shows separation of particles with different compressibility (bead vs. HL-60 cell). The uncertainty of the initial position is 60 μm in the simulation. The distributed areas of the simulation data are due to the initial position perturbation. For the “15 μm” super-posed area in panel A, the “31 dbm” super-posed area in panel B and the “Bead” super-posed area in panel C, the areas converge to a line due to the domination of acoustic force. A schematic representation of an exemplary taSAW device is shown in Panel C (right).

FIG. 5 Is a graph showing separation distance as a function of particle size and input power. Lateral migration of particles of distinct sizes are investigated under different input power. Each data point represents 5 repeats.

FIG. 6 Panel A shows optimization of the inclined angle for maximum separation efficiency using numerical simulation. Dependence of the separation distance between two microbeads with diameters of 10 μm and 4 μm on the inclined angle θ for different power levels (20 dbm, 25 dbm, and 30 dbm) at the outlet. Panel B shows dependence of the separation distance (between MCF-7 cancer cells and white blood cells) and the inclined angle θ for different power levels (25 dbm, 35 dbm, and 45 dbm) at the outlet. MCF-7 cancer cells and white blood cells have different diameters (20 μm vs. 12 μm), different compressibilities (4.22×10⁻¹⁰ ·Pa⁻¹ vs. 3.99×10⁻¹⁰ ·Pa⁻¹) and different densities (1,068 kg/m3 and 1,019 kg/m3).

FIG. 7 Panel A is a schematic showing the experimental and simulation trajectories of 10 μm polystyrene particles in the taSSAW separation device. The shaded line is calculated from the simulation model. Scale bar represents 650 μm. Panels B and C are enlarged images showing the match between the simulation trajectory and the upstream main experimental trajectory (Panel B) and downstream locations (Panel C) in the taSSAW field, respectively. Scale bar in panel B represents 160 μm. The power input was 38 dBm; flow rate was 170 μL/min; IDTs length was 1 cm.

FIG. 8 Shows diagrams of a theoretical investigation of multiple design parameters for high-throughput separation using a taSSAW. Panel A shows the relationship between tilt angle and separation distance under different flow rates. For higher flow rates, the optimum tilt angle becomes smaller. When flow rates are larger than 75 μL/min, the separation distance becomes smaller even at the optimum tilt angle. The power input is fixed at 35 dBm. Panel B shows the relationship between the length of IDTs and the separation distance under a fixed power input (35 dBm) and flow rate (75 μL/min).

FIG. 9 Shows a diagram of the relation between tilted angle and separation distance under different power inputs. To reach the same separation distance, smaller tilted angles required much less power input. The flow rate is fixed at 75 μL/min.

FIG. 10 Panel A shows cancer cell separation from human white blood cells. A panorama formed by stacked images shows that a single MCF-7 cell was pulled out from the stream of leukocytes. The superposed areas are simulation results for the same parameters. Panel B shows fluorescent images of cells collected from the outlet before separation. Erythrocyte-lysed human blood sample was spiked with MCF-7 cells, and the original cancer cell concentration was 10%. Dots indicated by arrows, labeled red, (indicating a positive for EpCAM) are recognized as MCF-7 cells while the unlabeled dots (positive to CD45) are indicators for leukocytes. Panel C shows fluorescent images of cells collected from the outlet after separation. Panel D shows florescene images of MCF-7 after exposure to EpCAM. Panel E shows florescene images of MCF-7 after exposure to CD45. Panel F shows florescence images of MCF-7 after exposure to DAPI were used to identify MCF-7 from the leukocytes. Panel G shows a composite of the three fluorescence images of panels D, E, and F. Panel H shows purity and recovery rate of collected cancer cells in this experiment.

FIG. 11 Shows diagrams of cancer cell separation performance under different power inputs at a 20 μL/min flow rate. Panel A shows the relationship between power input and separation performance for separation of MCF-7 from WBCs. Panel B shows The relationship between power input and separation performance for separation of HeLa from WBCs. Both cancer cell lines (MCF-7 and HeLa) showed a similar relationship between the power input and separation performance. Higher power input would result in better cancer cell recovery rates and lower WBCs removal rates, and vice versa. Error bars in Panels A and B represent the relative counting error. The number of cells (n) passing through collection channel and waste channel were counted, respectively. n>100 for cancer cells, while n>350 for WBCs.

FIG. 12 Shows cell viability and proliferation assays. Panel A shows experimental results for MCF-7 Cell viability, and Panel B shows proliferation tests for four different samples: 1) positive control (no SAW treatment), 2) cells passing through the device with SAW off, 3) cells passing through the device with SAW on, and 4) negative control. Cell viability imaging was also carried out for positive control (shown in Panel C), cells passing through the device with SAW off (shown in Panel D), and cells passing through the device with SAW on (shown in Panel E).

FIG. 13 Shows a schematic of surface acoustic wave based cancer cell separation.

FIG. 14 Shows separation of Hela cell from WBCs before (Panel A) and after (Panel B) turning on SAW. Smaller dots, shown in the expanded image of Panel C, are white blood cells (WBCs), whereas larger black dots, shown in the expanded image of Panel D, are Hela cells. Sample flow rate is 1.2 mL/h.

FIG. 15 Shows separation performance under different power input.

FIG. 16 Shows fluorescence and white light images of collection channels and waste channels after separating rare Hela cells from white blood cells. Green fluorescent cells are Hela cells. The images showed the removal of WBCs from Hela cells.

FIG. 17 Shows viability and proliferation tests after SAW treatment. Panel A shows the % viability of cells treated with SAW (SAW) and control cells not treated with SAW (Control). The condition for SAW-treated group is the same as the actual separations. Cells are stained with Calcein Am and PI to differentiate live and dead cells for the SAW group (Panel B) and the Control group (Panel C). Dead cells are considered dead if they are PI-positive (indicated as PI by arrows). Panel D shows images of the proliferating cells at various time points (14 hours (h), 38 h, 87 h, 111 h, 135 h, and 159 h), demonstrating that cells proliferate after being exposed to SAW treatment.

FIG. 18 Panel A shows an image of an exemplary SSAW device consisting of a piezoelectric substrate with a pair of segmented IDTs (S-IDT) and a PDMS channel on it. The inset of Panel A shows a Zoomed-in image of the S-IDTs. Panel B shows an illustration of the phase-shifting SSAW fields generated by two SIDTs. Particles experience consistent lateral displacements as passing through the SSAW fields. PN: pressure node. Panel C shows stacked fluorescent images of the particle trajectories at different flow rates with and without phase-shifting SSAW fields.

FIG. 19 Panel A shows a mechanism of particle/cell separation in phase-shifting SSAW fields (PN: Pressure node, AN: Pressure Antinode, A: Half wavelength, d: Distance of displacement). Panel B shows a typical stacked fluorescent image of particle separation using phase-shifting SSAW fields generated by S-IDT.

FIG. 20 Shows immunofluorescence images for the identification of CTCs in blood samples from breast cancer patients. Four channels, DAPI, CK8, 18, CD45 and ER, were examined. The MCF-7 cell was used as the positive control, showing a staining pattern of DAPI+/CK8, 18+/CD45−/ER+. CTCs were identified as they showed a staining pattern DAPI+/CK8, 18+/CD45−. In contrast, WBCs showed a staining pattern DAPI+/CK8, 18-/CD45+. Scale bar represents 4 μm.

### DETAILED DESCRIPTION OF THE INVENTION

Separation of cells is a critical process for studying cell properties, disease diagnostics, and therapeutics. Methods for cell sorting using acoustic waves as a means to separate cells on the basis of their size and physical properties in a label-free, contactless, and biocompatible manner are described herein. The separation sensitivity and efficiency of currently available methods for separating cells has been limited. The methods of the invention have provided for the first time the ability to separate low abundance cells from a fluid of mixed cell populations using acoustic waves.

Methods for separating cells, e.g., cancer cells or other low abundance cells from a mixed population of cells have been achieved according to aspects of the invention. The methods involve flowing a fluid sample containing a mixed population of cells through a channel, where the cells of the fluid sample are exposed to a surface acoustic wave (SAW). The SAW causes the cells to separate into discrete flowing streams. One flowing stream has the majority of low abundance cells and the other flowing stream has the majority of the remaining cells. The independent cell streams can then be collected into separate chambers at the end of the flow channel. The method described herein, can be used to separate out low-abundance cells. For instance cells that account for less than 10% of the total mixed population of cells may be removed from a tissue sample using the methods of the invention.

As used herein, a “mixed population” of cells refers to a mixture of cells having at least one low abundance cell and at least one other cell. The low abundance cells may be, for instance, cancer cells. A “cancer cell”, as defined herein, is a cell characterized by abnormally regulated growth and/or proliferation. A cancer cell includes a cell undergoing early, intermediate or advanced stages of multi-step neoplastic progression as previously described (H. C. Pitot (1978) in “Fundamentals of Oncology,” Marcel Dekker (Ed.), New York pp 15-28), including a pre-neoplastic cell (i.e., hyperplastic cell and dysplastic cell) and a neoplastic cell. Both cancer cells from established cancer cell lines and cancer cells obtained from patients (e.g. from a biopsy) are contemplated. Cells may be obtained as “samples” by a variety of techniques such as phlebotomy, aspiration, biopsy, brush biopsy, cystoscopy, endoscopy, lavage, pleural effusion, lumbar puncture, swabbing, and brushing.

In some embodiments, the inventive methods are used to separate low abundance cells from a mixed population of cells. In some embodiments, the low abundance cells make up less than 10% of the cells in the mixture. In some embodiments, the low abundance cells make up less than 9%, less than 8%, less than 7%, less than 6%, less than 5%, less than 4%, less than 3%, less than 2%, less than 1%, less than 0.1%, less than 0.001%, less than 0.0001%, less than 0.00001%, less than 0.000001%, less than 0.0000001%, less than 0.00000001%, or less than 0.000000001% of the cells in a mixed population of cells.

In some embodiments, the inventive methods are used to separate low abundance cells from a mixed cell population with a separation efficiency of at least 80%. Separation efficiency may be determined by dividing the number of low abundance cells collected in a collection channel by the sum of the low abundance cells collected in both the collection channel and waste channel. In some embodiments, the separation efficiency of low abundance cells from a mixed cell population using the inventive methods is at least 80%, at least 85%, at least 90%, at least 95%, at least 99%, or at least 100%.

In some embodiments, a fluid sample comprising a mixed cell population flows through the channel at a rate of at least 1mL of sample per hour. Other embodiments 90/min (0.54 mL/hr), 2 μl/min (0.12 mL/hr). To maintain high separation sensitivity and efficiency, a throughput of about 2 μ/min (0.12 mL/hr) for cells (10,000-20,000 cells/min) may be sufficient.

The low abundance cells may be separated from the mixed cell population in a manner that maintains high cell integrity, viability and proliferation. Cell integrity may refer to membrane integrity, proliferative integrity, organelle integrity, nuclear integrity, genetic integrity, intracellular signaling integrity or cell functions. It should be appreciated that methods for assessing cell integrity³⁷, cell viability and proliferation are commercially available and well known in the art. For example, the WST-1 cell viability test (Roche, Nutley, N.J.) and the BrdU Cell Proliferation ELISA (Roche, Nutley, N.J.) may be used to test the cells' viability and proliferation. In some embodiments cell viability and proliferation are assessed by measuring metabolic activity and DNA synthesis. In some embodiments, cell viability is assessed by staining. Cell integrity may be based on an assessment of both viability and proliferative capacity.

In some embodiments, the low abundance cells are cancer cells that are not cultured cancer cells. A “cultured cancer cell” refers to a cancer cell that has been maintained and/or propagated in vitro for ten or more passages. In some embodiments, the cultured cancer cells are note from an established cell line. For example, a cultured cancer cell lines may be HL-60 human promyleocytic leukemia cells, MCF-7 human breast cancer cells, HeLa human cervical cancer cells, or another cell from an established cell line.

The cancer cells separated according to the methods of the invention may be circulating tumor cells (CTCs). The term “circulating tumor cell” (CTC) is intended to mean any cancer cell that is found in a subject's sample. Typically CTCs have been exfoliated from a solid tumor. As such, CTCs are often epithelial cells shed from solid tumors found in very low concentrations in the circulation of patients with advanced cancers. CTCs may also be mesothelial from sarcomas or melanocytes from melanomas.

The cells are from a biological sample of a subject. The term “biological sample” may be used generally to refer to any biological material which may be obtained from a subject. For example, the biological sample may be whole blood, plasma, tissue (e.g., normal tissue or tumor tissue), urine, feces, or cells. The biological sample typically is a fluid sample. Solid tissues may be made into fluid samples using routine methods in the art.

In some embodiments, the cells, such as CTCs may be separated from non-cancer cells in blood having from 1 to 100 cancer cells in one mL, 1 to 10 cancer cells/mL, 1 to 20 cancer cells/mL, 1 to 30 cancer cells/mL, 1 to 40 cancer cells/mL, 1 to 50 cancer cells/mL, 1 to 60 cancer cells/mL, 1 to 70 cancer cells/mL, 1 to 80 cancer cells/mL, 1 to 90 cancer cells/mL, 60 to 1000 cancer cells/mL, 60 to 100 cancer cells/mL, 60 to 250 cancer cells/mL, 60 to 500 cancer cells/mL, 60 to 750 cancer cells/mL, 60 to 900 cancer cells/mL, 100 to 250 cancer cells/mL, 100 to 500 cancer cells/mL, 100 to 750 cancer cells/mL, 100 to 900 cancer cells/mL, 100 to 1000 cancer cells/mL, 250 to 500 cancer cells/mL, 250 to 750 cancer cells/mL, 250 to 900 cancer cells/mL, 250 to 1000 cancer cells/mL, 500 to 750 cancer cells/mL, 500 to 900 cancer cells/mL, 500 to 1000 cancer cells/mL, 750 to 900 cancer cells/mL, 750 to 1000 cancer cells/mL, or 900 to 1000 cancer cells/mL of fluid. In other embodiments, the cells such as CTCs may be separated from non-cancer cells in fluid having less than 100 cancer cells/mL, less than 90 cancer cells/mL, less than 80 cancer cells/mL, less than 70 cancer cells/mL, less than 60 cancer cells/mL, less than 50 cancer cells/mL, less than 40 cancer cells/mL, less than 30 cancer cells/mL, less than 20 cancer cells/mL, or less than 10 cancer cells/mL.

CTCs and other cells may be separated from a mixed population of cells in a non-invasive manner. The term “non-invasive” referred to herein means that the process of obtaining a biological sample from a subject does not require a major surgical procedure. For example, a non-invasive manner of collecting a biological sample may include, but is not limited to a venipuncture, a swab, a collection of fluid such as sputum or urine or a biopsy or other similar procedure.

The methods can also be used to separate label free cells from a mixture. A fluid biological sample containing cells may be exposed to a surface acoustic wave (SAW) to separate the label-free cells from the other cells, e.g., non-cancer cells, in a biological sample. The term “label-free” means that the cells are not labelled, marked, stained, or otherwise identified by another means to aid their separation prior to or during their separation by the inventive methods disclosed herein. It should be appreciated that the methods for separating label-free circulating tumor cells, disclosed herein, rely on the physical properties of the CTCs themselves.

The methods of separating cells from a mixture may be useful for a variety of purposes. The methods, for instance, may be used for monitoring the disease progress of a patient. The CTCs may be separated from a biological sample of a patient using the SAW based methods, described herein, and analyzed to assess disease progress. A number of ways for assessing disease progress by analyzing CTCs are well known in the art. For example, enumeration of (CTCs) in the peripheral blood of cancer patients has been associated with both disseminated disease and a higher risk of cancer progression. Several lines of evidence confirm that the detection of CTCs represents a new and reliable tool to predict the outcome of patients. Furthermore, the enumeration of CTCs at different time points during treatment has proven to be a reliable surrogate marker of treatment response and a potential alternative for non-invasive therapy monitoring. In some embodiments, the number of circulating tumor cells of a patient are determined at least every day, every 7 days, every 14 days, every 28 days, every month, every 2 months, every 4 months, every 6 months, every 9 months, every year, every 2 years, every 5 years, every 10 years, or every 20 years to monitor disease progress.

Additionally, the methods may be used for determining the genetic mutations of a cancer patient. For example, CTCs may be separated from a biological sample of a patient using the SAW based methods, described herein, and analyzed to identify genetic mutations. Mutation analysis of isolated CTCs may be accomplished by any known method (e.g., sequencing, polymorphisms). Mutations are commonly identified by PCR of DNA followed by direct sequencing of the amplified DNA. Isolated CTCs may be assessed for a number of mutations, including single base substitutions, insertions, deletions, duplications and translocations. Detection methods for these categories of mutations are well known in the art and may include but are not limited to sequencing, fluorescence in situ hybridization (FISH), single nucleotide polymorphism (SNP) analysis or karyotyping.

A single base substitution (point mutation or single nucleotide polymorphism) occurs when a base is exchanged for another (for example, C to T) in the DNA sequence. Depending on the substitution, this type of mutation can change the encoded amino acid (missense mutation) to produce a different protein or an incomplete protein (nonsense mutation) which can lead to a diseased state. An example of this type of mutation is sickle cell disease. Insertions and deletions occur when a single or multiple base-pairs are incorporated or deleted from a DNA sequence. This type of mutation can create frameshifts that cause the mRNA sequence to be not read properly by the translational machinery. Frameshifts can have devastating consequences. An example of this type of mutation is Huntington disease. Duplications occur when a section of the genome is duplicated. This type of mutation can create overexpression. An example of this type of mutation is high blood pressure. Translocations may occur when a portion of one chromosome is transferred to a nonhomologous chromosome. An example of this type of mutation is Burkitt lymphoma.

Methods for diagnosing cancer may also be achieved using the methods described herein. For example, CTCs may be separated from a biological sample of a patient using the SAW based methods and analyzed to diagnose the cancer. Methods for diagnosing cancer in CTCs may include, but are not limited to morphological analysis, mutational analysis, analysis of cell type/origin, or analysis of cancer biomarkers. A cancer biomarker, as defined herein, refers to a substance or process that is indicative of the presence of cancer in the body. A biomarker may be a molecule secreted or expressed by a CTC. Genetic, epigenetic, proteomic, glycomic, and imaging biomarkers can be used for cancer diagnosis and prognosis using CTCs. A number of biomarkers used to diagnose specific cancer types are well known in the art. For example AFP (Liver Cancer), BCR-ABL (Chronic Myeloid Leukemia), BRCA1/BRCA2 (Breast/Ovarian Cancer), BRAF V600E (Melanoma/Colorectal Cancer), CA-125 (Ovarian Cancer), CA19.9 (Pancreatic Cancer), CEA (Colorectal Cancer), EGFR (Non-small-cell lung carcinoma), HER-2 (Breast Cancer), KIT (Gastrointestinal stromal tumor), PSA (Prostate Specific Antigen) (Prostate Cancer), S100 (Melanoma), and many other biomarkers may be used to diagnose cancer using isolated CTCs.

The methods disclosed herein may also be used for predicting the therapy outcome of a patient. For example, CTCs may be separated from a biological sample of a patient using the SAW based methods, described herein, and analyzed to predict therapy outcome. It should be appreciated that the inventive methods, described herein, including quantifying CTCs, determining genetic mutations of CTCs, and diagnosing cancer by analyzing biomarkers in CTCs may be used to predict the therapy outcome of a patient. It is well known that biomarkers can be useful in determining the aggressiveness of an identified cancer as well as its likelihood of responding to a given treatment. This may be because CTCs exhibiting particular biomarkers may be responsive to treatments tied to that biomarker's expression or presence. For example, prognostic biomarkers include, but are not limited to, elevated levels of metallopeptidase inhibitor 1 (TIMP1), a marker associated with more aggressive forms of multiple myeloma; elevated estrogen receptor (ER) and/or progesterone receptor (PR) expression, markers associated with better overall survival in patients with breast cancer; HER2/neu gene amplification, a marker indicating a breast cancer will likely respond to trastuzumab treatment; a mutation in exon 11 of the proto-oncogene c-KIT, a marker indicating a gastrointestinal stromal tumor (GIST) will likely respond to imatinib treatment; and mutations in the tyrosine kinase domain of EGFR1, a marker indicating a patient's non-small-cell lung carcinoma (NSCLC) will likely respond to gefitinib or erlotinib treatment.

In some aspects the invention involves the use of a pair of segmented interdigital transducers (S-IDT) to make a high-resolution SSAW-based microfluidic apparatus capable of efficiently separating low abundance cells or particles or cells having similar properties to other cells in the mixture from fluid. With this S-IDT design, the generated SSAW fields inside the fluidic channel are divided into many discontinued, independent units along the flow direction. Each segmented SSAW field has a certain phase shift from the previous one. Different from the conventional acoustic based separation technologies, as the particles/cells pass through the phase-shifting SSAW fields, they will repeatedly experience acoustic radiation forces with different directions in the lateral direction. Therefore, even a small difference in flow trajectories can be magnified, resulting in significant separation of different particles/cells. The segments of the segmented interdigital transducers (S-IDT), described herein, may have any number of segments ranging from 5 to 30 segments (e.g., 5, 6, 7, 8, 9, 10, 11, 12, 13, 14, 15, 16, 17, 18, 19, 20, 21, 22, 23, 24, 25, 26,27, 28, 29 or 30 segments). The length of any of the segments of the S-IDT, described herein, may have a length ranging from 100 μm to 1000 μm. In some cases the S-IDT has 15 segments that are 25 um long.

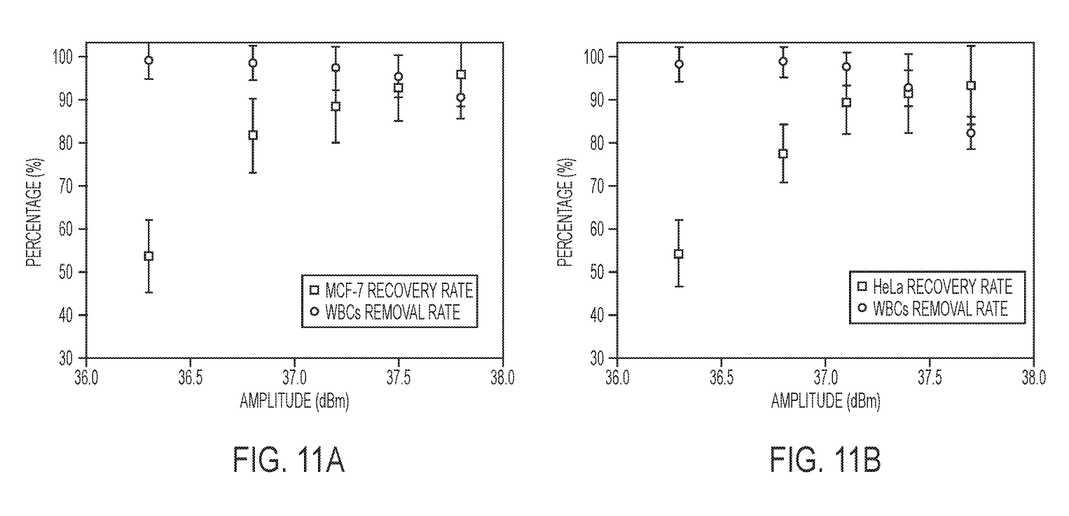

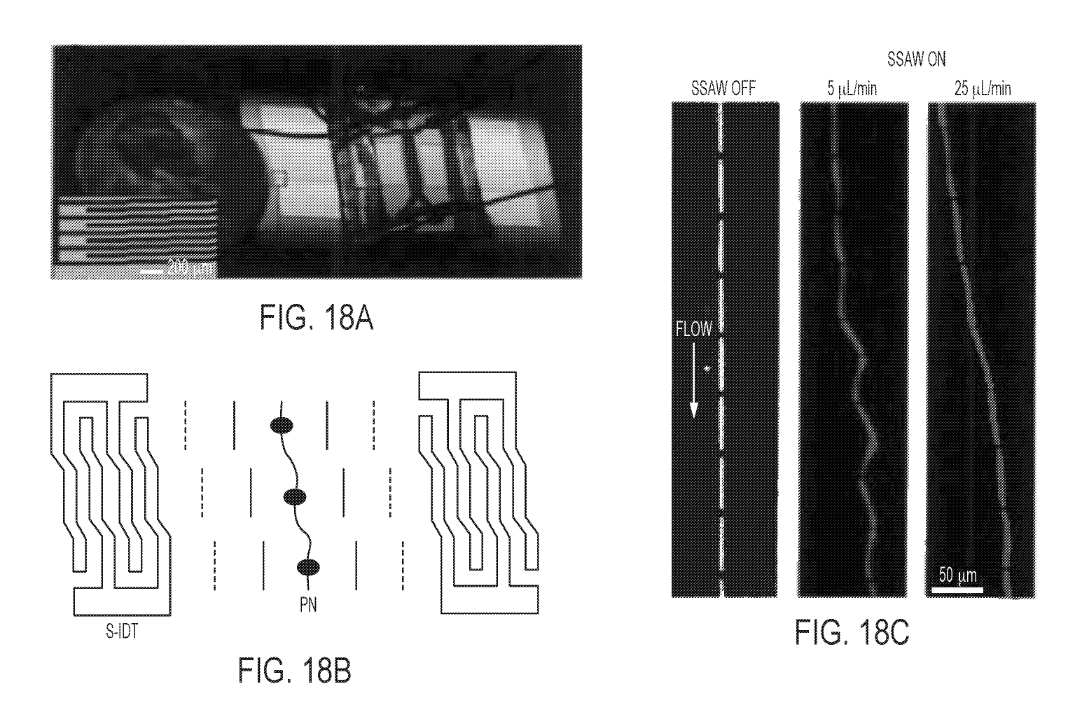

In some embodiments, the surface acoustic wave of the inventive method is generated by at least two interdigital transducers. An interdigital transducer (IDT, also referred to as an interdigitated transducer), is a device comprising two interlocking comb-shaped metallic coatings which are applied to a piezoelectric substrate. In some embodiments, the interdigital transducers (IDTs) are positioned to produce a SAW within a flow chamber. In some embodiments, the surface acoustic wave of the inventive method is generated by at least two segmented interdigital transducers (S-IDTs). A segmented interdigital transducer (SID), shown in FIG. 18, Panel A, consists of many small sections of parallel IDTs. Each section has a consistent displacement from the previous one in the lateral direction. The function of the S-IDT is to generate many discontinued, independent SAW fields in a flow chamber along the flow direction. Each field has a certain phase shift from the previous one (FIG. 11, Panel B). In the phase-shifting SAW fields, cells have a chance to experience acoustic radiation forces from different directions and move to different pressure nodes. Therefore, the separation distance can be increased with more S-IDT segments, and consequently the separation resolution can be significantly increased.

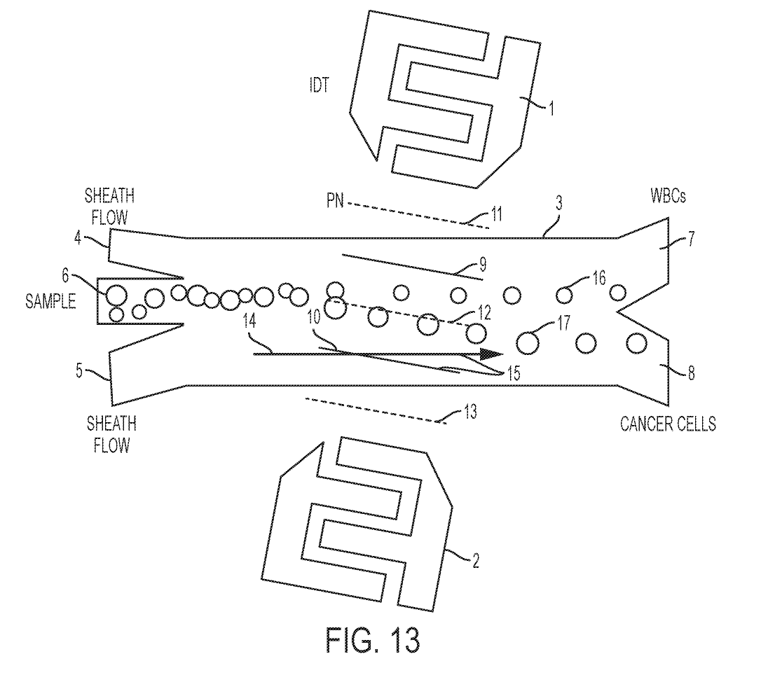

The surface acoustic wave generators (e.g., IDTs or S-IDTs), 1 and 2 of FIG. 13, can produce a surface acoustic wave (SAW), which may generate pressure forces (nodes and anti-nodes) in a fluid sample flowing through a fluid channel 3. For example, the channel may pass between a pair of SAW generators. The SAW may be said to have a SAW direction, which lies along the line 12 in FIG. 13. That is, the SAW direction is a direction aligned with the generally linear anti-nodes 9 and 10 and nodes 11, 12, and 13 of the SAWs. As will be clear to those of skill in the art, SAWs have nodes and anti-nodes, which may be positioned differently than shown and may move over time. However, they will still occur at the angle 15 indicated with respect to the flow channel direction 14. The SAW direction may be at an angle with respect to the flow and channel direction. In some embodiments the SAW direction is at a non-oblique angle (e.g., at a parallel or a right angle) to the direction of flow in the channel.

In some embodiments, the SAW direction is at an oblique angle 15 to the direction of flow 14 in the channel 3 in FIG. 13. As known to those of skill in the art, oblique defines an angle between 0 and 90 degrees and between 90 and 180 degrees, not including 0, 90 or 180. In some embodiments, the SAW direction ranges from 0 to10 degrees to the direction of flow in the channel. In other embodiments, the SAW direction ranges from 10 to 15 degrees to the direction of flow in the channel. In other embodiments, the SAW direction ranges from 15 to 30 degrees to the direction of flow in the channel. In other embodiments, the SAW direction ranges from 30 to 45 degrees to the direction of flow in the channel. In other embodiments, the SAW direction is at about a 15 degree angle, a 30 degree angle, or a 45 degree angle to the direction of the flow in the channel.

Herein, a SAW that is at an oblique angle to the direction of flow in a channel may be referred to herein as a tilted-angle SAW. In the tilted-angle SAW design, a particle in a fluid experiences both the acoustic radiation force and the laminar drag force. The competition between these two forces determines the position of the particle and defines its movement along the pressure nodal lines, which lie across the channel at a particular angle of inclination to the flow. As a result, the migration distance of the particle along the direction perpendicular to the flow could be a few times or tens of times the acoustic wavelength, depending on the geometry of the channel. This migration distance is significantly higher than that of the traditional acoustic separation approaches. The ability of tilted-angle SAW to achieve much larger separation distances leads to better separation sensitivity. In addition, the tilted-angle SAW design utilizes multiple pressure nodal lines for separation instead of only one pressure nodal line in the conventional acoustic separation designs. Since there are many parallel pressure nodal lines lying across the flow, target particles that escape from one pressure nodal line can be trapped again by the neighboring nodal line and be separated from the nontarget particles. This multiple-node design also produces higher separation efficiency.

The acoustic power emitted from the SAW generators may affect how cells align with the pressure nodal lines (nodes and anti-nodes). In some embodiments, the SAW has an acoustic power ranging from 19 dBm to 31dBm. It should be appreciated that dBm (sometimes referred to as dBmW or Decibel-milliwatts) is an abbreviation for the power ration in decibels of the measured power referenced to one milliwatt. In some embodiments, the SAW has an acoustic power of about 19 dBm, 23 dBm, 27 dBm, or 31 dBm. For an input power of 31 dBm, the cells may only align with one pressure nodal line. For input powers of 19 dBm, 23 dBm, or 27 dBm, cells may cross two, three, four, five or more pressure nodal lines which may increase separation distance and separation efficiency.

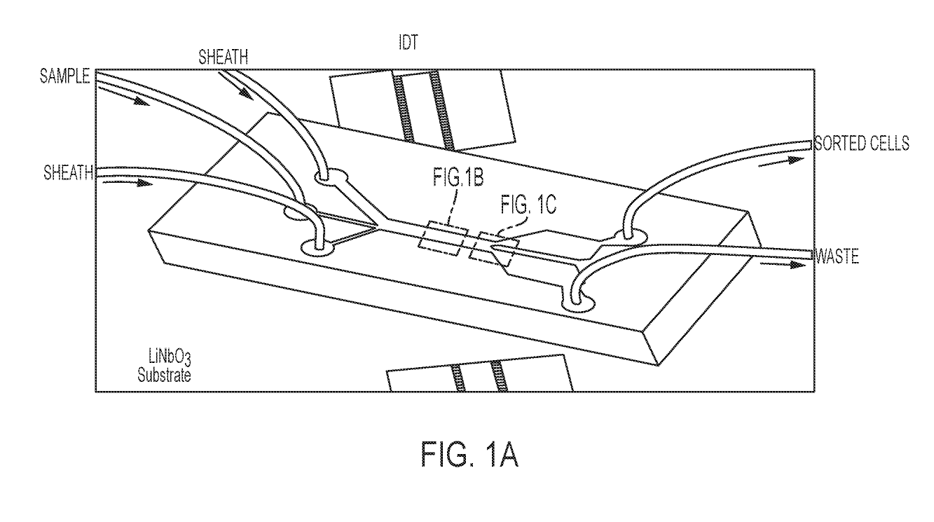

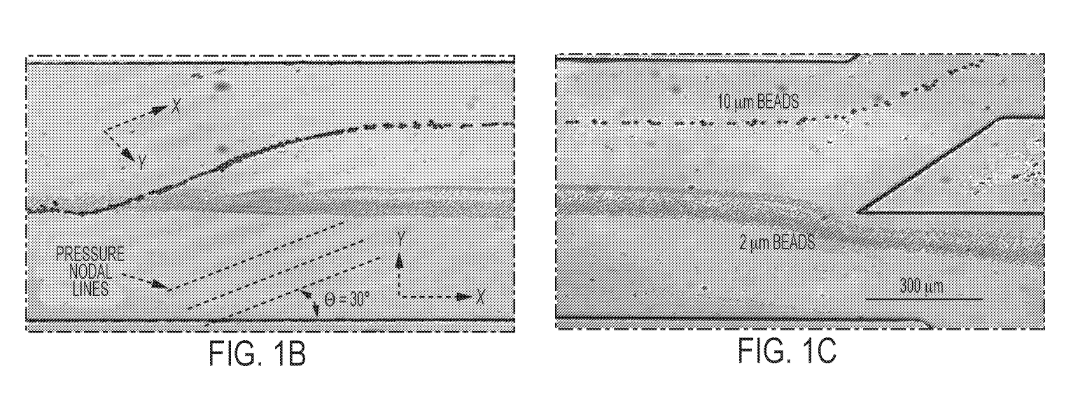

Aspects of the disclosure relate to an apparatus for sorting cells from a mixed population of cells. One example of the apparatus is shown in FIG. 13. A channel 3, on a surface, may be disposed in a piezoelectric substrate between a pair of spaced apart surface acoustic wave (SAW) generators 1 and 2. A preferred feature of the disclosed apparatus is that the acoustic wave generators are segmented interdigitated transducers (S-IDTs), shown in FIG. 18, Panels A and B. The S-IDTs generate SAW fields inside the fluidic channel that are divided into many discontinued, independent units along the flow direction FIG. 18, Panel B. Each segmented SAW field has a certain phase shift from the previous one. Different from the conventional separation technology, as the cells pass through the phase-shifting SAW fields, they will repeatedly experience acoustic radiation forces with different directions in the lateral direction. Therefore, even a small difference in flow trajectories can be magnified, resulting in significant separation of different cells in the lateral direction. The SAWs, depicted in FIG. 1, Panel B, for clarity, have nodes indicated by dotted lines at 11, 12, and 13 and anti-nodes indicated by solid lines at 9 and 10. It should be appreciated that the nodes and anti-nodes generated by a S-IDT are phase-shifted as depicted in FIG. 18, Panel B.

A fluid containing a mixed population of cells may be introduced into the center inlet port 6 at the inlet end of the channel 3. The apparatus may have two additional inlet ports 4 and 5 which may be used to introduce a buffer flow. The outlet end of the channel 3, may have two outlet ports 7 and 8 that may receive cells 16 and 17 from a cell mixture. The SAW may be said to have a SAW direction, which lies along the line 12 in FIG. 13. That is, the SAW direction is a direction aligned with the generally linear anti-nodes 9 and 10 and nodes 11, 12, and 13 of the SAWs. As will be clear to those of skill in the art, SAWs have nodes and anti-nodes, which may be positioned differently than shown and may move over time. However, they will still occur at the angle 15 indicated with respect to the flow channel direction 14. It should be appreciated that the phase-shifted pressure nodes created by an S-IDTs (FIG. 18, Panel B) may occur at an angle with respect to the flow channel as similarly depicted in FIG. 13.

In certain embodiments the SAW direction of the apparatus is at a non-oblique angle (e.g., at a parallel or a right angle) to the direction of flow in the channel. In some embodiments, the SAW direction of the apparatus is at an oblique angle 15 to the direction of flow 14 in the channel 3 in FIG. 13. As known to those of skill in the art, oblique defines an angle between 0 and 90 degrees and between 90 and 180 degrees, not including 0, 90 or 180. In some embodiments, the SAW direction ranges from 0 to10 degrees to the direction of flow in the channel. In other embodiments, the SAW direction ranges from 10 to 15 degrees to the direction of flow in the channel. In other embodiments, the SAW direction ranges from 15 to 30 degrees to the direction of flow in the channel. In other embodiments, the SAW direction ranges from 30 to 45 degrees to the direction of flow in the channel. In other embodiments, the SAW direction is at about a 15 degree angle, a 30 degree angle, or a 45 degree angle to the direction of the flow in the channel.

The acoustic power emitted from the SAW generators may affect how cells align with the pressure nodal lines (nodes and anti-nodes). In some embodiments, the SAW generator of the apparatus has an acoustic power ranging from 19 dBm to 31dBm. In other embodiments, the SAW has an acoustic power of about 19 dBm, 23 dBm, 27 dBm, or 31 dBm. For an input power of 31 dBm, the cells from a fluid mixture of cells may only align with one pressure nodal line. For input powers of 19 dBm, 23 dBm, or 27 dBm, cells may cross two, three, four, five or more pressure nodal lines which may increase separation distance and separation efficiency.

In certain embodiments, the surface of the apparatus is a piezoelectric substrate that supports the electrodes of the surface acoustic wave generators. It should be appreciated that the surface of the apparatus may form a wall of the channel 3. The apparatus may be considered a microfluidic device, which has a microchannel that may have at least one dimension less than 1 mm.

Another aspect of the disclosure relates to methods of separating cells or particles based on their physical properties, (e.g., compressibility, size and density). The methods involve flowing a sample containing a mixed population of cells or particles through a channel, where the cells or particles of the fluid sample are exposed to a surface acoustic wave (SAW). The SAW causes the cells to separate into discrete flowing streams depending on their physical properties. The first flowing stream may contain cells or particles having the same, or a similar, value for a physical property and the second flowing stream may contain cells or particles having the same, or a similar, value for a physical property that is different from the cells or particles of the first flowing stream. The independent cell or particle streams can then be collected into separate chambers at the end of the flow channel, thereby separating the cells or particles having different physical properties.

Cells or particles having different sizes can be separated using the methods described herein. For example, a fluid containing a mixture of polystyrene beads having a diameter of either 9.9 μm or 7.3 μm are exposed to a SAW as they flow through a channel. The beads having a diameter of 9.9 μm separate out into one flowing stream while the beads having a diameter of 7.3 μm separate out into a separate flowing stream. The beads from each flowing stream may be collected in separate outlet channels. The methods may be used to separate cells or particles having at least a 2.6 μm difference in diameter. For example the methods may be used to separate cells or particles having a difference in diameter of at least 3 μm, at least 4 μm, at least 5 μm, at least 6 μm, at least 8μm, at least 10 μm, at least 15 μm, at least 20 μm, at least 25 μm, at least 30 μm, at least 35 μm, at least 40 μm, at least 45 μm, or at least 50 μm. In other embodiments, the methods may be used to separate cells or particles having at less than a 2.6 μm difference in diameter.

Cells or particles having at least a 27% difference in diameter may be separated from one another. For example, the methods may be used to separate cells or particles having a difference in diameter of at least 30%, at least 40%, at least 50%, at least 60%, at least 70%, at least 80%, at least 90%, at least 100%, at least 150%, or at least 200%. In other embodiments, the methods may be used to separate cells or particles having at less than a 27% difference in diameter.

It should be appreciated that the mixture of cells or particles may contain more than two populations of cells having different sizes. For example, the mixed population of cells or particles may have at least three, four, five, six, seven, eight, nine, or ten populations of cells or particles of different size that may be separated into distinct flowing streams using the inventive methods. In some cases, the mixed population of cells or particles may have more than 10 populations of cells or particles of different size. It should also be appreciated that these flowing streams may be collected in any number of outlet ports. For example a fluid sample containing particles of 4 different sizes may be separated into four flow streams (referred to as flow stream 1, 2, 3, and 4) using the inventive methods. In this example, the four flow streams may be collected in four separate outlet ports (referred to as outlet port 1, 2, 3 and 4, respectively).

The methods, described herein, allow for the efficient separation of cells or particles based on size. The methods may be used to separate cells or particles from a mixed cell population with a separation efficiency of at least 80%. Separation efficiency may be determined by dividing the number of cells or particles collected in a collection channel by the sum of the cells or particles collected in both the collection channel and waste channel. The separation efficiency of cells or particles from a mixed population using the methods may be at least 80%, at least 85%, at least 90%, at least 95%, at least 97%, at least 99%, or at least 100%. In other embodiments, the separation efficiency of cells or particles from a mixed population may be less than 80%.

The inventive methods may be used to separate cells or particles having a different compressibility or density. Compressibility, as defined herein, is a measure of the relative volume change of a substance as a response to a pressure change. Methods for measuring the compressibility of cells or particles are well known in the art³⁵ (e.g., on-chip measurements of cell compressibility via acoustic radiation). As an example, a fluid may contain a mixture of two populations of cells or particles having similar or identical densities and sizes but different compressibilities. In some embodiments the methods may be used to separate cells or particles having a difference in compressibility of at least 0.23×10⁻¹⁰ Pa⁻¹, at least 0.30×10⁻¹⁰Pa⁻¹, at least 0.40×10⁻¹⁰ Pa⁻¹, at least 0.60×10⁻¹⁰ Pa⁻¹, at least 0.80×10⁻¹⁰ Pa⁻¹, at least 1.0×10⁻¹⁰Pa⁻¹, at least 1.5×10⁻¹⁰ Pa⁻¹, at least 2.0×10⁻¹⁰ Pa⁻¹, at least 5.0×10 ⁻¹⁰ Pa⁻¹, at least 10.0×10⁻¹⁰ Pa⁻¹, at least 25.0×10⁻¹⁰ Pa⁻¹, or at least 50.0×10⁻¹⁰ Pa⁻¹. In some embodiments the methods may be used to separate cells or particles having a difference in compressibility of less than 0.23×10⁻¹⁰ Pa⁻¹. In some embodiments the methods may be used to separate cells or particles having a difference in compressibility of at least 5%, at least 10%, at least 20%, at least 30%, at least 40%, at least 50%, at least 60%, at least 70%, at least 80%, at least 90%, at least 100%, at least 120%, at least 150%, at least 175%, or at least 200%. In some embodiments the methods may be used to separate cells or particles having a difference in compressibility of less than 5%.

Cells or particles having a different density may be separated from one another. It is well known to those skilled in the art that density, as defined herein, of a cell is its mass per unit volume which may be expressed in kilograms per cubic meter (kg/m³). Methods for measuring the density of cells or particles are well known in the art³⁶. As an example, a fluid may contain a mixture of two populations of cells or particles having similar or identical compressibilities and sizes but different densities. In some embodiments the methods may be used to separate cells or particles having a difference in density of at least 49 kg/m³, at least 60 kg/m³, at least 80 kg/m³, at least 100 kg/m³, at least 150 kg/m³, at least 200 kg/m³, at least 400 kg/m³, at least 600 kg/m³, at least 800 kg/m³, or at least 1000 kg/m³. In some embodiments the methods may be used to separate cells or particles having a difference in density of less than 49 kg/m³. In some embodiments the methods may be used to separate cells or particles having a difference in density of at least 5%, at least 10%, at least 20%, at least 30%, at least 40%, at least 50%, at least 60%, at least 70%, at least 80%, at least 90%, at least 100%, at least 120%, at least 150%, at least 175%, or at least 200%. In some embodiments the methods may be used to separate cells or particles having a difference in density of less than 5%.

It should be appreciated that the mixture of cells or particles may contain more than two populations of cells having different compressibilities or densities. For example, the mixed population of cells or particles may have at least three, four, five, six, seven, eight, nine, or ten populations of cells or particles of different compressibilities or densities that may be separated into distinct flowing streams using the inventive methods. In some cases, the mixed population of cells or particles may have more than 10 populations of cells or particles of different compressibilities or densities. It should also be appreciated that these flowing streams may be collected in any number of outlet ports. For example a fluid sample containing particles of 4 different compressibilities or densities may be separated into four flow streams (referred to as flow stream 1, 2, 3, and 4) using the inventive methods. In this example, the four flow streams may be collected in four separate outlet ports (referred to as outlet port 1, 2, 3 and 4, respectively).

The inventive methods, described herein, allow for the efficient separation of cells or particles based on compressibility or density. In some embodiments, the inventive methods are used to separate cells or particles from a mixed cell population with a separation efficiency of at least 80%. Separation efficiency may be determined by dividing the number of cells or particles collected in a collection channel by the sum of the cells or particles collected in both the collection channel and waste channel. In some embodiments, the separation efficiency of cells or particles from a mixed population using the inventive methods may be at least 80%, at least 85%, at least 90%, at least 95%, at least 97%, at least 99%, or at least 100%. In other embodiments, the separation efficiency of cells or particles from a mixed population may be less than 80%.

The methods relating to the velocity of flow, the angle between the SAW and flow direction, the alternative IDTs used to generate a SAW, or any apparatus, described herein, may be alternative embodiments of the methods of separating cells or particles based on their compressibility or density.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1

### Cell Separation Using Tilted-Angle Standing Surface Acoustic Waves

### Design and Characterization

FIG. 1, Panel A illustrates the structure and the working mechanism of the tilted-angle standing surface acoustic wave (taSSAW) separation device. A polydimethylsiloxane (PDMS) microfluidic channel was bonded in between a pair of identical interdigital transducers (IDTs) coated on a piezoelectric substrate. The microfluidic channel consists of three inlets and two outlets. The pair of IDTs were deposited in a parallel arrangement with respect to each other, and aligned at a specific angle with respect to the channel and flow direction. A radio frequency (RF) signal was imposed at each IDT to generate two identical surface acoustic waves (SAWs). These two SAWs propagate toward each other and interfere to form a SSAW in between the IDTs located within the PDMS microchannel. Such a SSAW generates parallel pressure nodal and anti-nodal lines at a particular angle to the flow direction, and is termed “taSSAW”.

The acoustic radiation force, generated from the pressure distribution within the microfluidic channel, pushes the suspended particles toward the pressure nodal or anti-nodal lines in the taSSAW field, depending on the physical properties (such as volume, compressibility, and density) of particles and medium. Particles were first injected through the center inlet channel and then were hydrodynamically focused in the main channel by two sheath flows before entering the taSSAW working region.

When a particle enters the SSAW working region, it experiences an acoustic radiation force, a drag force, a gravitational force, and a buoyancy force. With the small dimensions of the microfluidic channels, the gravitational and the buoyancy forces have similar magnitude but opposite direction, and are almost balanced. Thus, the behavior of particles in the microfluidic channel can be characterized by examining the drag force (Fd) and the acoustic radiation force (Fr). The acoustic radiation force tends to confine particles at the pressure nodal lines while the drag force induced by the flowing fluid pushes particles forward along the flow direction. When a mixture of particles with various sizes passes through the taSSAW region, they all experience acoustic radiation force and drag force. The acoustic radiation force dominates over the drag force for larger particles and results in a lateral displacement. Conversely, the drag force dominates over the acoustic force for smaller particles, resulting in little lateral displacement.

Using the taSSAW approach, separation of 10 μm and 2 μm polystyrene beads with 99% separation efficiency is demonstrated. The separation process can be found in the stacked images in FIG. 1, panels B and C, where the large particles (10 μm) were collected in the upper outlet channel while the small ones (2 μm) accumulated in the lower outlet. In the exemplary device, a number of pressure nodal lines fall within the channel at 30° to the flow direction, as depicted in FIG. 1, Panel B. While the small particles tend to traverse those multiple pressure nodal lines, the large particles will be confined within the nodal lines and migrate along a path line at 30° with respect to the flow of the smaller particles. Since the velocity of each individual particle varies, some larger particles may escape from the first pressure node; however, they would eventually be captured at subsequent neighboring nodes and continue to migrate across the channel. The multiple pressure nodal lines along the path of the particle stream significantly improved the separation efficiency and sensitivity compared to the previously demonstrated SSAW-based particle separator (27), in which there was only one pressure nodal (or antinodal) line in the channel. Note that the keys for high-efficiency separation in the taSSAW mechanism is the angle between pressure nodal lines and the flow, as well as the device operation power, which will be explored in later sections.

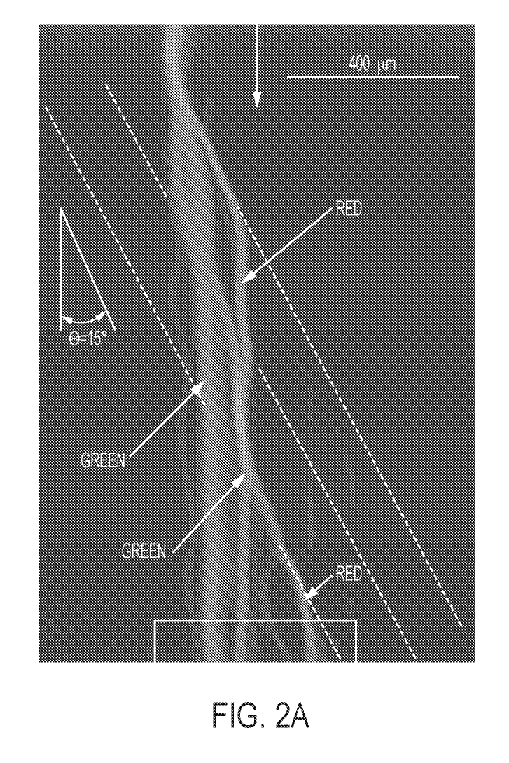

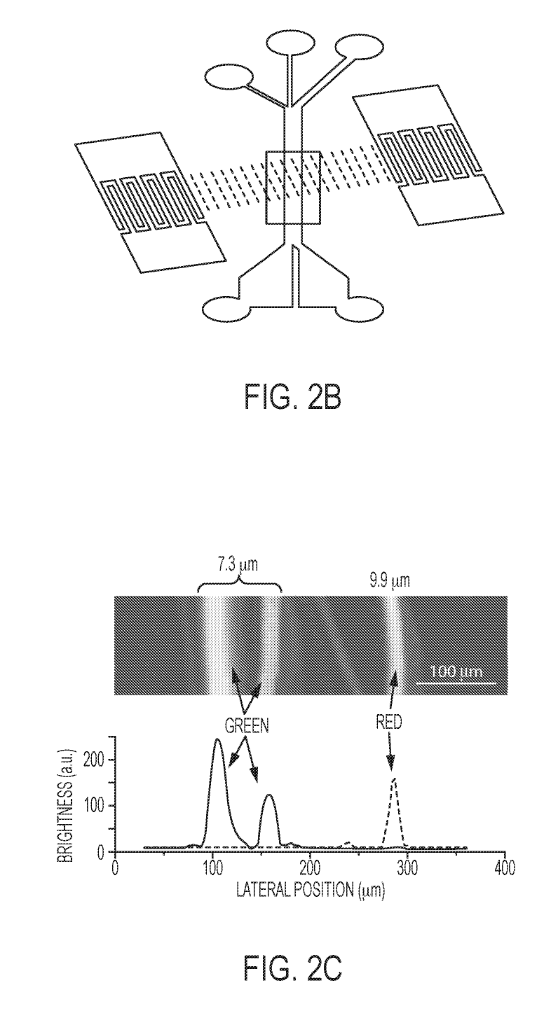

With the taSSAW design, separation of particles with small size differences also becomes feasible. Next high-efficiency separation of 7.3 and 9.9 μm particles was demonstrated. An aqueous solution with fluorescent polystyrene beads with diameters of 9.9 μm (arrows labeled red) and 7.3 μm (arrows labeled green) was injected into the main channel of the device at a flow velocity of ˜1.5 mm/s. The angle between the SSAW pressure nodal lines and the flow direction is set at 15 degrees for this particular experiment. The length of the overlapping IDT electrode fingers is designed to be 4 mm, corresponding to the length of the pressure nodal lines in the SSAW working region. The input working frequency (˜19.4 MHz) is determined by the period of the IDT fingers which is 200 μm. The main channel is 75 μm high and 1,000 μm wide. The mixed beads were focused upstream before entering the SSAW working region in the channel (FIG. 2, Panels A and B). The input power was set at 25 dBm. The fluorescence intensity of beads was profiled near the outlet channel to indicate the mean distribution of the bead positions. Downstream of the SSAW working region, the 9.9 μm beads had a lateral migration of ˜130 μm more than the 7.3 μm beads. By splitting the outlet channel near the center of the peak labeled green and peak labeled red (at about ˜220 μm of the lateral position in FIG. 2, Panel B), a separation efficiency of ˜100% for 9.9 μm beads and ˜97% for 7.3 μm beads was achieved. The two peaks in distribution of the smaller beads could be caused by the parabolic velocity distribution of the fluid in the vertical direction and imperfect particle focusing before entering the SSAW working region.

### Numerical Simulation of Particle Separation

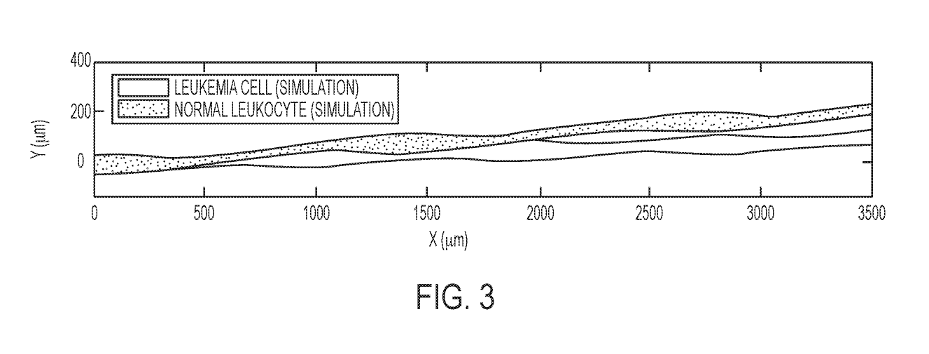

Parametric studies of the key variables in the design of the device would improve the efficiency of separating microbeads of similar sizes and isolating cancer cells from normal cells. In order to better understand the mechanism of taSSAW-based particle separation, an analysis of the trajectory of a particle was performed in a rectangular channel flow under an acoustic radiation force field using numerical simulations. In particular, the effects of acoustic power, particle size, and particle compressibility on particle trajectory were studied, and compared the numerical results with corresponding experimental results. Details about numerical simulation of particle separation are listed in FIG. 3.

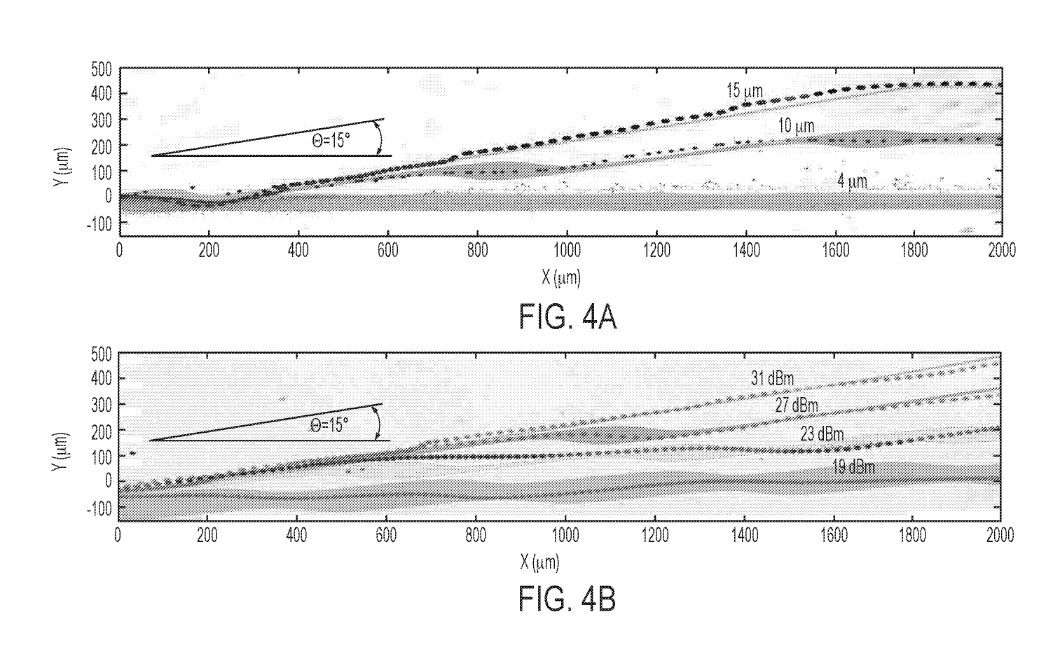

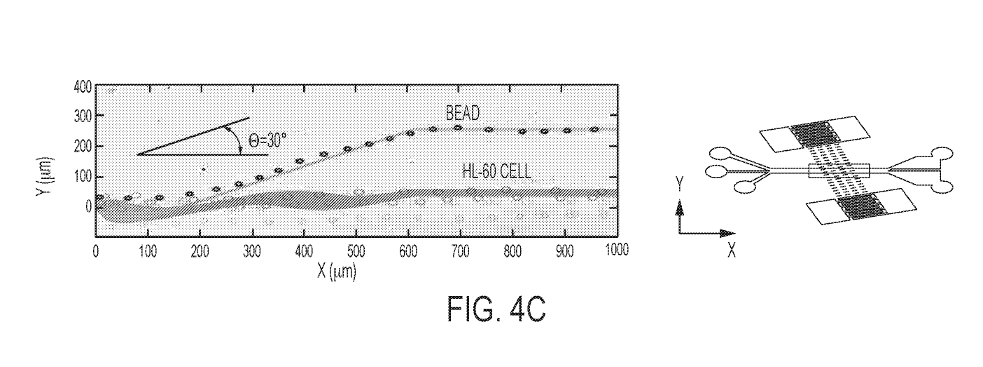

First, the particle size effect was examined and considered three different diameters: 15, 10, and 4 μm. A comparison between the simulation data and the experimental results is shown in FIG. 4, Panel A. The particle separation process was captured digitally (10 frames/s) and is presented as stacked images in FIG. 4, Panel A. The black dots are trajectories of particles with different sizes. The areas with different shadings are trajectory ranges predicated by numerical simulations for three different particle sizes [15 μm (top), 10 μm (middle), and 4 μm (bottom)]. The initial positions of particles ahead of the taSSAW working region are distributed in a range of 60 μm in Y direction. For all three different particle sizes, the predicted trajectories in simulations match well with the experiment. For particles with a diameter of 15 μm, they only lie in one pressure nodal line and form a single line (the top line in FIG. 4, Panel A). This is because the acoustic radiation force for 15 μm particles is very strong and overcomes the initial disturbed vertical positions of particles. For diameter of 10 μm, particles cross two pressure nodal lines. In the transition between pressure nodal lines, the simulations show spread areas, while between these transitions, the particle trajectories converge to narrow lines, which are the pressure nodal lines as shown by the middle area in FIG. 4, Panel A. For diameter of 4 μm, since the acoustic radiation force is much weaker than flow-induced drag force, the particles almost follow the original undisturbed streamlines as shown by the bottom area in FIG. 4, Panel A.

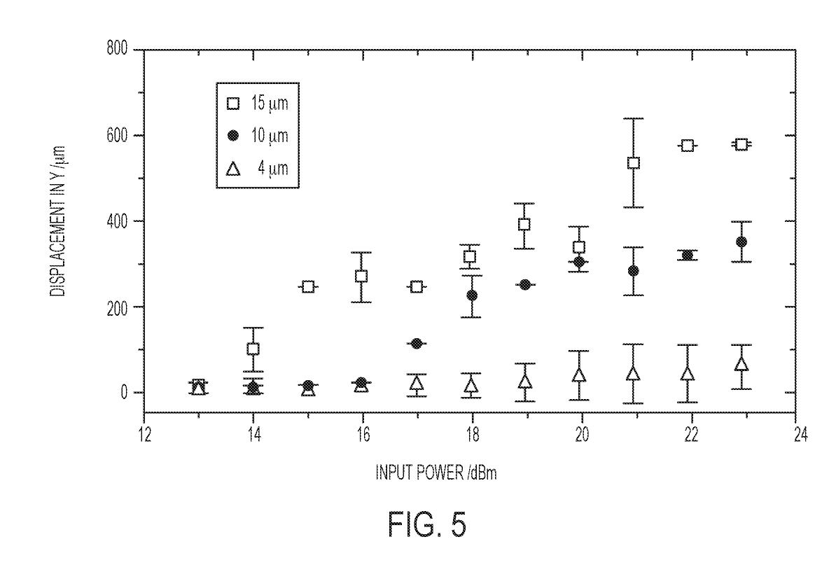

Second, the particle trajectories under different acoustic powers was studied, (19 dBm, 23 dBm, 27 dBm, and 31 dBm) and the diameter of the bead was chosen for this purpose to be 15 μm. The simulation results (areas with different shades in FIG. 4, Panel B) are consistent with the experimental results (black dots in FIG. 4, Panel B). For an input power of 31 dBm, the particles only stay in one pressure nodal line (top area); for input powers of 27, 23, and 19 dBm, the particles cross two, three, and five pressure nodal lines, respectively (second from top, third from top, and bottom in FIG. 4, Panel B). The simulation results again match well with experimental results. A series of tests were carried out to explore the overall effects of size and power on the particle trajectories. The trajectories of particles with various sizes under different powers are shown in FIG. 5.

Finally, the influence of the particle compressibility was studied. Particle separation has been demonstrated by applying magnetic, dielectrophoretic, optical, and hydrodynamic forces (6, 7, 19, 20). None of these methods, however, is capable of effectively separating particles on the basis of their compressibility difference. To explore the unique capability of the taSSAW-based method, particle separation based on differences in compressibility was carried out. HL-60 is a human promyelocytic leukemia cell line with average diameter, density and compressibility of approximately 15 μm, 1.075 kg m⁻³ and 4×10⁻¹⁰ Pa⁻¹, respectively. Polystyrene beads (˜15 μm in diameter, with density ˜1.05 kg m⁻³, and compressibility ˜2.16×10^{—10} Pa⁻¹) were mixed with HL-60 cells in a ratio of 1:4. They have similar densities and sizes but different compressibilities. In this case, inclination angle, θ=30°, a SAW wavelength^{λ=300μm}, and flow rate=9 μL/min were used. FIG. 4, Panel C shows that the simulation results once again closely match the experimentally observed particle trajectories. The small black dots in FIG. 4, Panel C are the trajectory of polystyrene beads in experiments, while the large grey dots are HL-60 cells. The top and bottom shaded areas in FIG. 4, Panel C are the trajectories for polystyrene beads and HL-60 cells, respectively, as predicted by numerical simulations.

### Optimization of the Angle of Inclination by Numerical Simulations

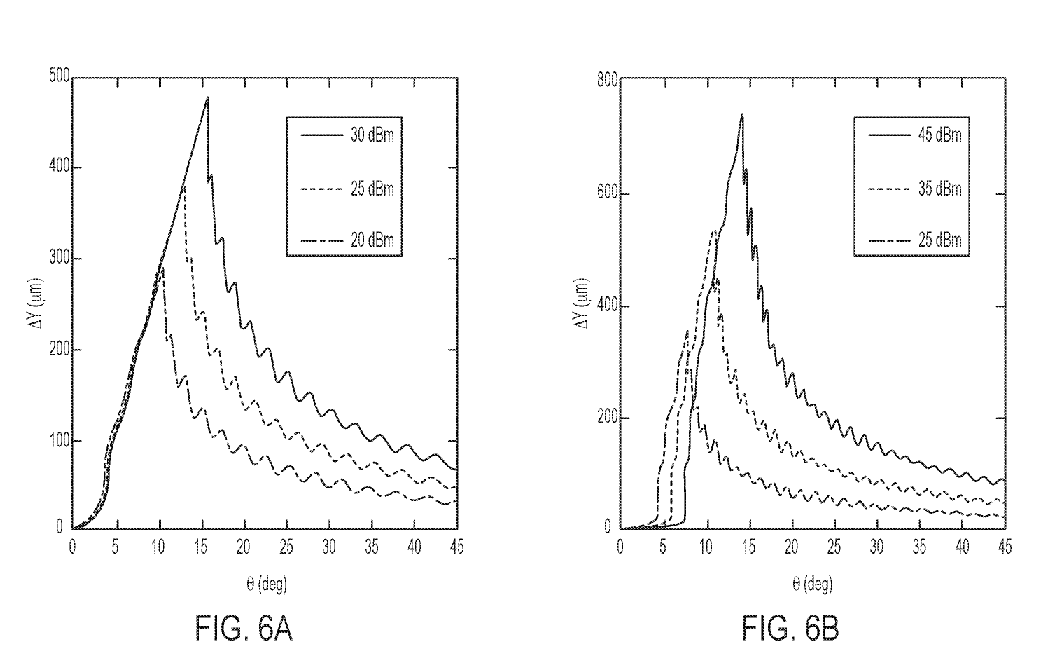

To further improve the separation efficiency, its dependence on the angle of inclination θ by numerical simulations was studied. For example, to achieve the maximum separation distance AY in the Y direction between two microbeads with diameters of 10 μm and 4 μm at the outlet, AY as a function of θ at different power levels was plotted (FIG. 6, Panel A). It shows AY that increases almost linearly when θ increases from zero degree to a higher value (depending on power levels) between 10° to 15°, and it drops significantly when θ increases to 45°. In addition, there are small oscillations in the dependence of AY on θ due to the increasing number of pressure nodal lines in the path of particles. For the power of 30 dBm, the separation distance ΔY (˜500 μm) with an inclined angle of 15° is twice as that (˜250 μm) with 10°. For different power levels, the initial increases of ΔY with θ overlap, and the maximum separation distance increases linearly with the power magnitude. To demonstrate the ability of this method to successfully separate cancer cells from healthy human white blood cells (WBCs), numerical simulations to find the optimal angle of inclination to maximize the separation efficiency between these two types of cells with different sizes (20 μm vs. 12 μm), different compressibilities (4.22×10⁻¹⁰ Pa⁻¹ vs.3.99×10⁻¹⁰ Pa⁻¹), and different densities (1,068 kg/m³ vs.1,019 kg/m³) were carried out. Since AY increases with increasing power, it was found that for the maximal operating power (45 dBm), the maximal separation ΔY can be achieved at an inclination angle of 15°. In distinct contrast to the case of the microbeads (FIG. 6, Panel A), cell separation leads to a noticeably different response: the initial increase of ΔY is slow when θ is small, and there is an abrupt increase after θ reaches certain value, as shown in FIG. 6, Panel B. In addition, the initial trajectories are quite different at different power levels, while they are almost the same for the microbeads (FIG. 6, Panel A). After θ reaches the optimal angle, AY decreases significantly when θ increases to 45°.

### Numerical Simulation of Particle Separation

The behavior of particles in the microfluidic channel can be characterized by examining the drag force and the acoustic radiation force. The acoustic radiation force (Fᵣ) and the drag force (F_{d}) are expressed as

$$\begin{matrix}{F}_{r} = - ( \frac{π {p}_{0}^{2} {V}_{p} {β}_{w}}{2 λ} ) φ ( β , ρ ) sin ( 2 ky ) , & [ S1 ] \\ φ ( β , ρ ) = \frac{5 {ρ}_{p} - 2 {ρ}_{m}}{2 {ρ}_{p} + {ρ}_{m}} - \frac{{β}_{p}}{{β}_{m}} , & [ S2 ] \\ {F}_{d} = - 6 πμ {R}_{p} {u}_{r} , & [ S3 ]\end{matrix}$$

where p₀, λ, Vₚ, ρₘ, ρₚ, βₘ, βₚ, μ, Rₚ and uᵣ are the acoustic pressure, acoustic wavelength, volume of the particle, density of the medium, density of the particle, compressibility of the medium, compressibility of the particle, viscosity of the medium, radius of the particle, and relative velocity of the particle, respectively. In Eq. S 1, y is the coordinate as shown in FIG. 1, Panel B and k is the wavenumber of the standing acoustic wave. From these equations, one can see that the magnitude of the acoustic radiation force acting on a particle is a function of its volume, density, compressibility, and the power of RF signal applied to device. Hence, particles with differences in these properties will experience varying radiation force, and can be separated in a SSAW device.

The working mechanism using size-based particle separation was demonstrated. Eq. S2 describes the acoustic contrast factor, φ, which determines whether a particle moves to a pressure nodal or an anti-nodal lines: the particles will aggregate at pressure nodal lines when φ is positive and at pressure anti-nodal lines when φ is negative. Most solid particles and cells in aqueous solution have a positive φ, and move towards pressure nodal lines in a SSAW field. Eqs. S 1-S3 indicate that the drag force is proportional to the radius of the particle or cell and the acoustic radiation force is proportional to the volume. The acoustic radiation force tends to confine particles at the pressure nodal lines while the drag force induced by the flowing fluid pushes particles forward along the flow direction. When a mixture of particles with various sizes passes through the taSSAW region, they all experience acoustic radiation force and drag force. The acoustic radiation force dominates over the drag force for larger particles and results in a lateral displacement. Conversely, the drag force dominates over the acoustic force for smaller particles, resulting in little lateral displacement. In the taSSAW design, the pressure nodal lines align with the flow direction at an angle θ as shown in FIG. 1, Panel B. Since the flow field is a linear Stokes flow in this length scale, the velocity of the particle can be decomposed into an undisturbed channel flow velocity u_{f} and a velocity uᵣ due to the acoustic radiation force. The position of the particle in the coordinate system { x, y, z }, as shown in FIG. 1, Panel B, at the center plane of the height (z=0) is described by the following ordinary different equations (ODEs):

$$\begin{matrix}\frac{dx}{dt} = {u}_{f} cos θ , & [ S4 ] \\ \frac{dy}{dt} = {u}_{f} sin θ + {u}_{r} , & [ S5 ]\end{matrix}$$

where u_{f} is the fluid velocity in an infinitely long channel with a rectangular section (width: a, and height: b), uᵣ is the velocity of the particle due to the acoustic radiation force imposed by the standing surface acoustic wave, and t is time.

It is well known that the fluid velocity u_{f} in a rectangular channel with width a and height b is given by

$${u}_{f} = \frac{∇ P}{2 μ} {b}^{2} [ 1 - \frac{{z}^{2}}{{b}^{2}} + 4 {\sum }_{n = 1}^{∞} \frac{{( - 1 )}^{n}}{{α}_{n}^{3}} \frac{cosh ( {α}_{n} \frac{Y}{b} )}{cosh ( {α}_{n} \frac{a}{b} )} cos ( {α}_{n} \frac{z}{b} ) ] , \qquad [ S6 ]$$

where Z is the coordinate in the channel height direction and Y is the coordinate in the width direction in the {X, Y, Z } coordinate system aligned with flow direction as shown in FIG. 1, Panel B, μ is the fluid viscosity, ∇P is the pressure gradient, and

$${α}_{n} = ( n - \frac{1}{2} ) π .$$

The coordinates in two different systems {X, Y, Z } and {x, y, z } can be converted by a rotational transformation with an angle θ.

The velocity of the particle due to the acoustic radiation force uᵣ in Eq. S5 can be obtained based on the balance between acoustic radiation force Fᵣ and viscous drag force F_{d}, which is a function of uᵣ. The viscous drag force is given by Stokes law as shown in Eq. S3, and the acoustic radiation force is given by Eq. S1. Due to force balance Fᵣ=F_{d}, the velocity of the particle due to the acoustic radiation force uᵣ in Eq. S5 is given by

$${u}_{r} = \frac{π {R}_{p}^{2} {p}_{0}^{2} {β}_{w}}{9 λμ} φ ( β , ρ ) sin ( 2 ky ) . \qquad [ S7 ]$$

It is assumed that the particle is in the center of the channel in the vertical direction so that Z=z=0. Combining Eqs. S4-S7, one can predict the trajectory of the particle in {x, y } coordinate system by integrating the ordinary differential equations numerically (using, for example, Matlab, Mathworks, Natick, Mass.) with initial particle positions of x₀ and y₀. One can then obtain the trajectory of the particle in {X, Y } coordinate system by a rotation transformation of an angle θ.

The dimensions of the channel used in the simulation were: L (length)=4,000 μm, W (width)=1,000 μm, and H (height)=75 μm. The default angle of inclination was θ=15°, the default wave length, λ=200 μm, and the default flow rate=11 μL/min. At room temperature, the compressibility of water=4.6×10⁻¹⁰/Pa and the compressibility of the beads=2.16×10⁻¹⁰/Pa. The density of the water=997 kg/m³, the viscosity of water=0.001 Pa·s, the density of the beads=1050 kg/m³, and the density of the substrate=4650 kg/m³. The sound speed of the substrate=3,997 m/s. The frequency (f) of the wave ˜20 MHz. The initial position of the particle was varied about 50˜60 μm in the experiments in the Y direction, in order to consider a variation of 60 μm for Y₀ in the simulations. The acoustic pressure was evaluated as p₀=\sqrt{αP₁ρₛcₛ/A_{w}}, where P₁ is the power of the IDTs, ρₛ (taken to be 4,650 kg/m³ in this study) and cₛ (taken to be 3,997 m/s) are the density and the sound speed of the substrate, A_{w} is the working area given as the channel length multiplied by the distance between IDTs (4000 μm×2600 μm=1.04×10⁻⁵ m² in this study), and a is the power conversion efficiency in which the power of the IDTs converts to the acoustic pressure in the fluids. Since the power conversion efficiency is influenced by several factors, such as energy dissipation in the IDTs, in the substrate, and in the fluid, a was calibrated for each microfluidic device by matching the simulated particle trajectory with the experiment. Once calibrated, the same a was used for all simulations of the experiments done on this particular microfluidic device setup. The calibrated power conversion efficiency a in the experiments described herein is 10-20%. The default parameters indicated herein were used unless noted otherwise.

### Optimizing for High-Throughput Cell Separation

Due to the rareness of CTCs in a peripheral blood sample, it is necessary to process a large number of cells in order to detect the presence of tumor cells. Thus high-throughput separation is a critical requirement for any separation method targeting clinical CTC applications. In order to apply taSSAW to the separation of CTCs, the throughput had to be improved significantly. In this regard a parametric study was performed to determine the optimized design parameters such as the tilt angle and length of the IDTs, for high-throughput cell separation.

In order to find out the practical separation parameters for high-throughput cell separation, a simulation model was established that can numerically describe particle trajectories in the taSSAW microfluidic device. The model considered the effects of the acoustic radiation force, the hydrodynamic drag force, and the laminar flow profile in the microchannel on separation performance. A detailed description of the simulation model of particle separation is described above.

After calibrating the simulation model with 10-μm diameter polystyrene beads (FIG. 7), the dependence of separation distance on tilt angles under different flow rates was studied. To summarize the simulation conditions, the dimensions of the channel were 10,000 μm (L)×800 μm (W)×112 μm (H). The phase velocity of SAW is 3,997 m/s. The frequency of the SAW was ˜19.6 Mhz. The default wavelength of SAW was 200 μm. The default angle of inclination for beads calibration was 0=4.8°. For the liquid medium of water, the compressibility is 4.6×10⁻¹⁰1/Pa and its density is 997 kg/m³. The viscosity of water at room temperature is 0.001 Pa·s. The compressibility of PS beads is 2.16×10⁻¹⁰1/Pa, and the density of PS beads is 1,050 kg/m³. The density of LiNbO₃ substrate is 4,650 kg/m³. In the experiments, the variation in the initial position of PS beads was 20-30 μm in the direction of the channel width; as a result, in the simulations, a variation of 30 μm for the initial position of PS beads was considered. The acoustic pressure was estimated by p₀=\sqrt{αP_{I}ρₛcₛ/A_{w}}, where P_{I} is the input power to the IDTs, pₛ and cₛ are the density of the LiNbO₃ substrate and the phase velocity of SAW, respectively, A_{w} is the working area evaluated as the channel length multiplied by the distance between the two IDTs (10,000 μm×16,000 μm in this work), and α is the defined as the power conversion efficiency. The parameter α was calibrated by comparing the simulated trajectories of PS beads with the ones obtained in experiments. Its value was calibrated to be ˜0.15 for the device used in the experiments.

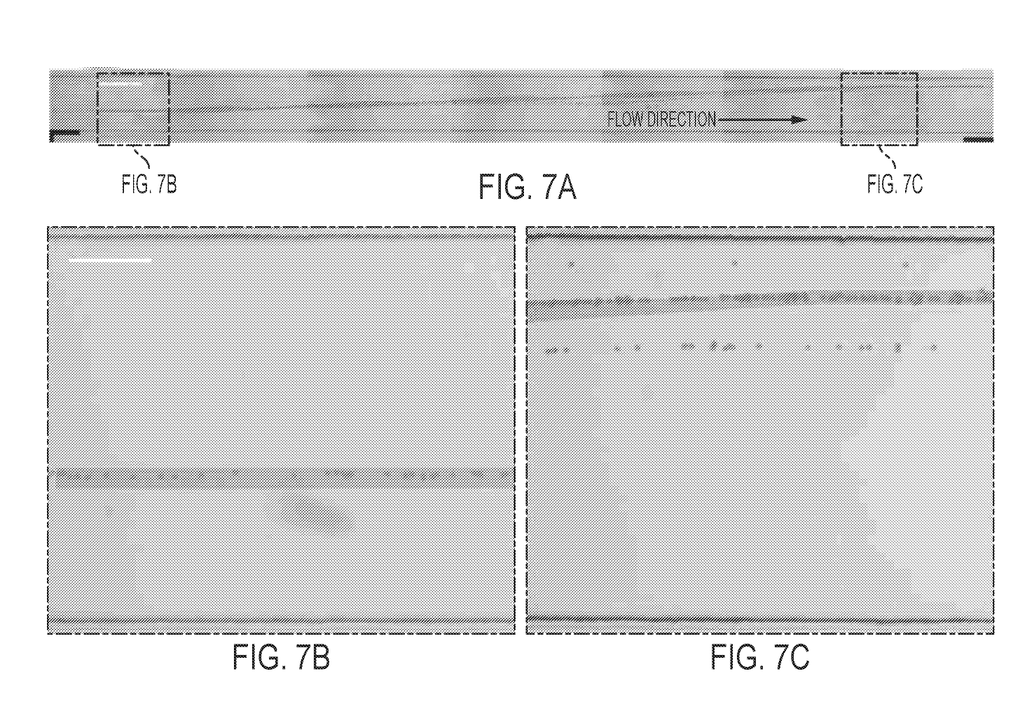

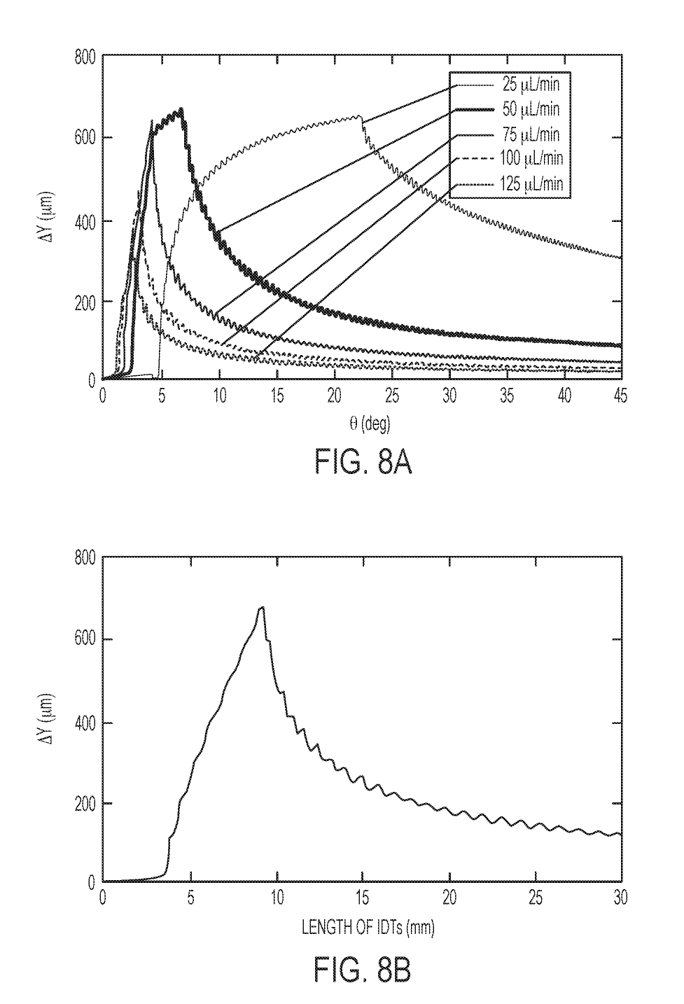

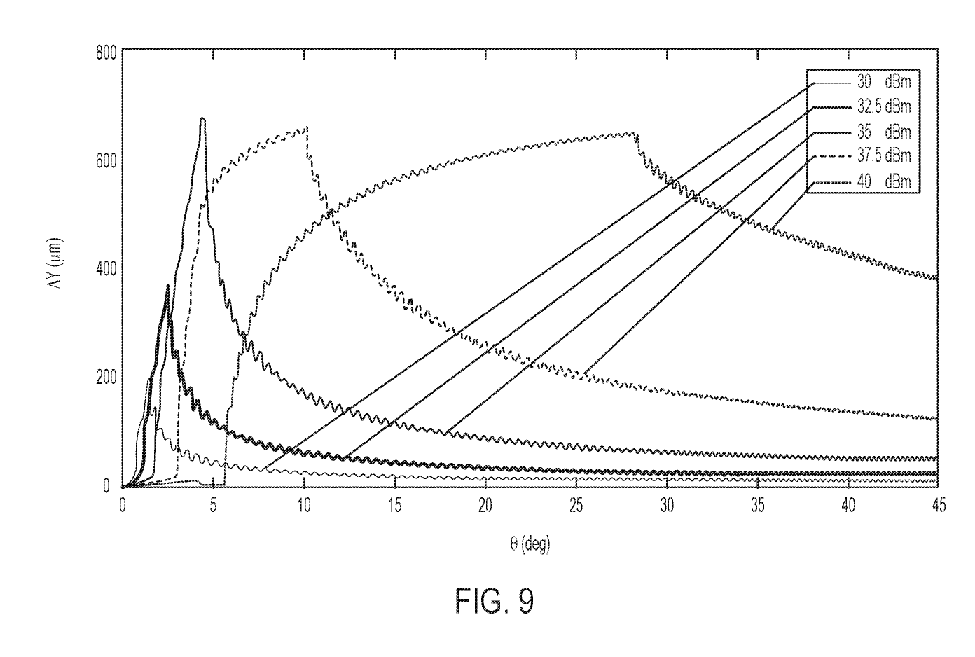

MCF-7 breast cancer cells and WBCs were used as the separation targets. The power input was set to 35 dBm. As shown in FIG. 8, Panel A, an optimum tilt angle for maximizing the separation distance can be found at each flow rate. The simulation results also indicate that as the flow rate increases, the tilt angle that achieves the highest separation distance decreases. The reason for this result may be attributed to the fact that smaller tilted angles allow longer travelling times between pressure anti-nodes and pressure nodes where the separation occurs. At high flow rates, the acoustic radiation force cannot dominate over the drag force. In this situation, a longer separation time enabled by the smaller tilt angle will be more advantageous for cell separation. At lower flow rates, the separation distance between cells is no longer limited by their exposure time in the taSSAW since the acoustic radiation force is dominant over the drag force. In this case, larger tilt angles would increase the separation distance. For the same flow rate, the simulation indicated that the optimum separation angle increased as the power increased (FIG. 9). This is because at higher power, the acoustic radiation force becomes more dominant. However, in practical situations, the applied power input cannot be too high as it could generate a level of Joule heating that could damage biological cells and/or device substrates.

Based on this parametric study, the optimum tilt angle was identified in order to achieve high-throughput cell separation. Under a practical power input (35 dBm), the separation distance for MCF-7 cells and WBCs at a 75 μL/min flow rate can reach ˜600 μm with a tilt angle of ˜5 deg., which is sufficient for successful separation at this throughput (FIG. 8, Panel A). When the gross flow rate is 75 μL/min, the sample flow rate can reach as high as 20 μL/min based on a 2.5:1 sheath-to-sample flow rate ratio, which means 1 mL of WBCs can be processed within one hour using this design. Although higher total flow rates are also possible at even small tilt angles, the separation distance could be compromised, which is not desirable. It is important to keep the separation distance as large as possible to compensate for potential variations in experimental conditions.

After optimizing the flow rate, power input, and tilt angle, the remaining design parameter that needs to be optimized is the length of the IDTs (i.e., the length over which the SSAW is applied). As discussed above, the travel time of the cells across the taSSAW field, which is dependent on the length of IDTs, is critical to the cell-separation outcome. Therefore, the length of the IDTs would inevitably have an impact on the efficiency of cell separation. In general, the longer the time that it takes the cells to traverse the taSSAW field, the longer is the separation distance. However, the larger IDTs imply a lower energy density for the same power input, thereby decreasing the primary acoustic radiation force. There should thus be an optimum IDT length that can balance these two competing factors. To find the theoretical optimum IDT length, the simulation was used to study the relationship between the separation distance and the length of the IDTs at a fixed flow rate and power input. FIG. 8, Panel B shows that the separation distance reaches a maximum at around an

IDT length of 8-10 mm. The decrease in the separation distance for shorter and longer IDTs lengths is caused by insufficient travel time through the taSSAW field and a lower energy density, respectively.

Separation of Cancer Cells from Human Healthy White Blood Cells

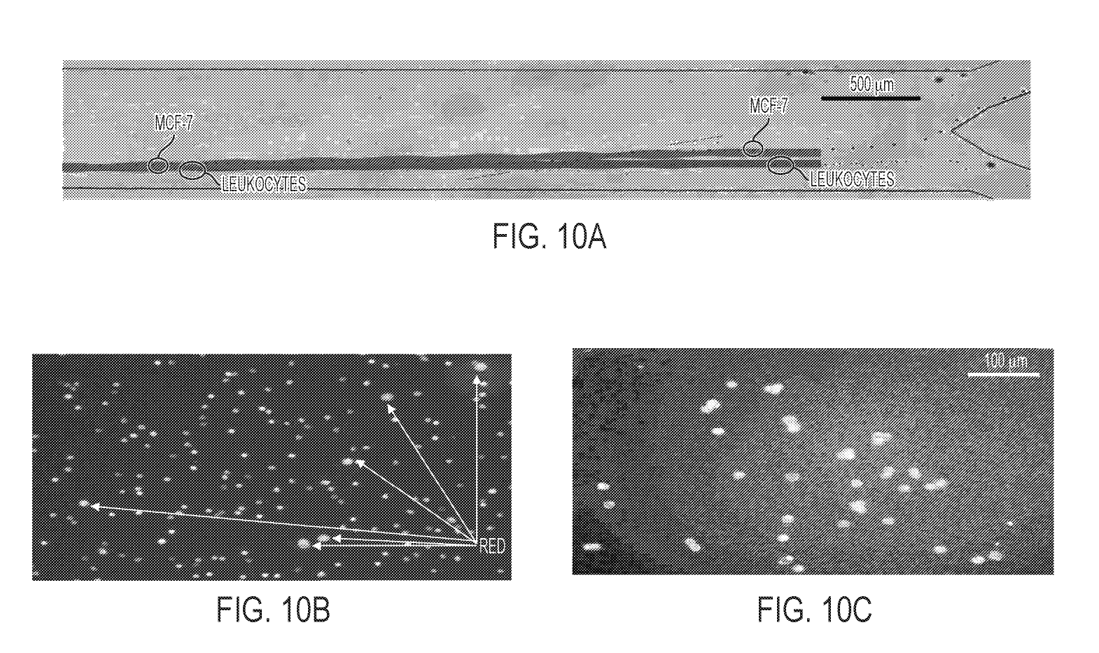

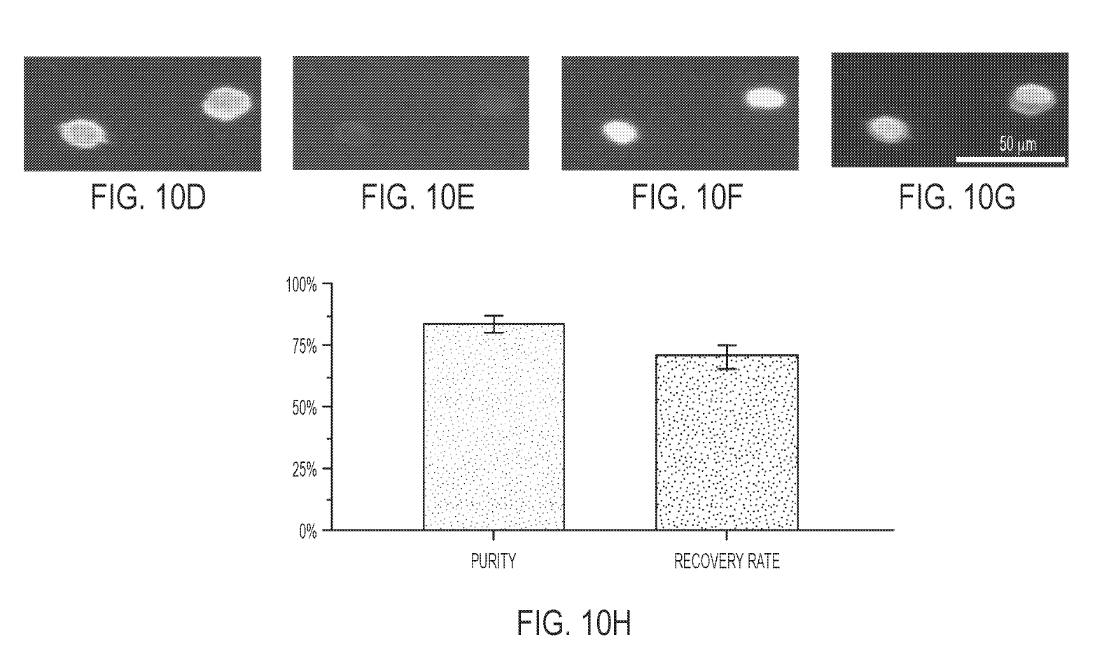

As a crucial step in isolating and analyzing circulating tumor cells for cancer diagnosis, the taSSAW device was used to separate MCF-7 cancer cells from normal leukocytes (white blood cells) using an optimized design, guided by the numerical simulation, with an angle of inclination of 15°. In this set of experiments, 1 mL human whole blood (Zen-bio, Durham, N.C.) was lysed using a red blood cell (RBC) lysis buffer (eBioscience, San Diego, Calif.), and the concentration of the collected leukocytes was measured to be ˜3×10⁶/mL. 1 mL of such erythrocyte-lysed blood sample was then mixed with 100 μL of cancer cells (˜3×10⁶ cells/mL) to achieve a cancer cell concentration of ˜10%. Here the MCF-7 cell (a human breast cancer epithelial cell line) was used as the cancer cell model. The mixed sample of fluorescently stained MCF-7 cells and normal leukocytes was then delivered into the taSSAW separation device through a syringe pump. Since MCF-7 cells are usually larger than leukocytes (as shown in FIG. 10, Panel A), when the cells entered the taSSAW working region, the ˜20 μm diameter MCF-7 cells were separated from the ˜12 μm diameter leukocytes. The process of isolating an MCF-7 from the leukocytes is shown in FIG. 10, Panel A, in which a series of time-lapsed images shows the position of one MCF-7 cell. As shown in FIG. 10, Panel A, the cell trajectories predicted by the numerical simulations match well with those obtained from the experiments. The simulation results also indicate that the compressibility difference between MCF-7 cells and leukocytes works against the size-difference-mediated separation. Specifically, the separation distance at the outlet is about 95 μm with both compressibility and size differences considered, but it is increased to 260 μm if the compressibility difference is removed in the numerical simulations by setting the compressibility of MCF-7 cells from 4.22×10⁻¹⁰Pa⁻¹ to the same value of normal leukocytes (3.99×10⁻¹⁰ Pa⁻¹). These results indicate that a small difference in compressibility can result in a significant increase of separation distance. Cancer cells and leukocytes were eventually collected at different outlets for subsequent identification. FIG. 10, Panels B and C are the fluorescence images of stained cells illustrating the cell distributions before and after separation. Here EpCAM (arrows labeled red), CD45 surface markers (non-indicated cells in Panel B), and a nuclear stain were used to determine the purity of the isolated MCF-7 cells. Epithelial cancer cells such as MCF-7 are positive to EpCAM and DAPI, and negative to CD45, while leukocytes are positive to CD45, and DAPI, and negative to EpCAM. Panel C shows that the separated cells are MCF-7 cells. FIG. 10, Panels D, E, and F show the fluorescence images of MCF-7 after exposure to EpCAM, CD45, and DAPI, respectively. FIG. 10, Panel G is a composite of the three fluorescence images. To quantitatively evaluate the capability of the device, the recovery rate and purity of cancer cell isolation was investigated. The recovery rate (%) and purity (%) for cell isolation are defined as the percentage of the number of isolated cancer cells over the number of spiked cancer cells, and that of the number of isolated cancer cells over the total number of collected cells, respectively. Under the current experimental conditions, a recovery rate of 71% and a purity of 84% was achieved, as shown in FIG. 10, Panel H. The flow fraction at the collection outlet (the upper outlet channel) significantly influences the purity and recovery. Logically, a higher fraction of flow into the collection stream results in a monotonic increase in the recovery rate and decrease in purity. For instance, additional experiments show that a 45% flow fraction into the collection stream yielded a purity of ˜98% and a recovery rate of ˜20%. However, a 60% flow fraction into the collection stream yielded a purity of ˜84% but increased the recovery rate to ˜71%.

Additional Demonstration of High-Throughput Separation of Cultured Cancer Cells from WBCs

Experimental verification of the high-throughput separation of cancer cells from WBCs was performed based on the optimized values obtained from the simulation. IDTs were fabricated with a tilt angle of 5 deg. and 1 cm length on a lithium niobate (LiNbO₃) piezoelectric substrate. A polydimethylsiloxane (PDMS) microfluidic channel with height and width of 110 μm and 800 μm, respectively, was bonded onto the substrate to form the separation device. For this, the input power is an important operating parameter. Higher input powers improve the recovery rate of cancer cells, while reducing the removal rate of WBCs, leading to decreased separation purity. Therefore it is necessary to obtain a profile of separation performance at different input powers. To evaluate the impact of varying the input power, the separation of MCF-7 and HeLa cells from WBCs was used as models. To facilitate the characterization of device performance, an abundant number of cancer cells mixed with WBCs was used.

FIG. 11, Panels A and B show the relationship between power inputs and cell-separation performance (the recovery rate and the removal rate of WBCs) for MCF-7 cells and for HeLa cells, respectively. Both MCF-7 cells and HeLa cells showed similar trends for power input dependence on separation performance. At lower input powers, the WBC removal rate could be maintained at ˜99%, but the recovery rate for cancer cells was only 60-80%. Using a higher power input can result in greater than a 90% of cancer cell recovery and ˜90% removal rate of WBCs. In particular, certain WBCs (e.g., monocytes) that have a larger size are more easily pushed by the acoustic field, resulting in a decrease in the WBC removal rate. The choice of the appropriate power input for cell separation thus depends on the outcome desired from optimization. If high sample purity is desired, a lower power input is preferred to ensure the highest removal rate of background cells. For CTC applications, the recovery rate of cancer cells is often more critical because of the inherent rarity of CTCs. For the following rare cell separation experiments, the higher input power values (˜37.5 dBm) were used to ensure a high recovery rate while maintaining ˜90% removal rate of WBCs.

### Tests on Cell Viability and Proliferation

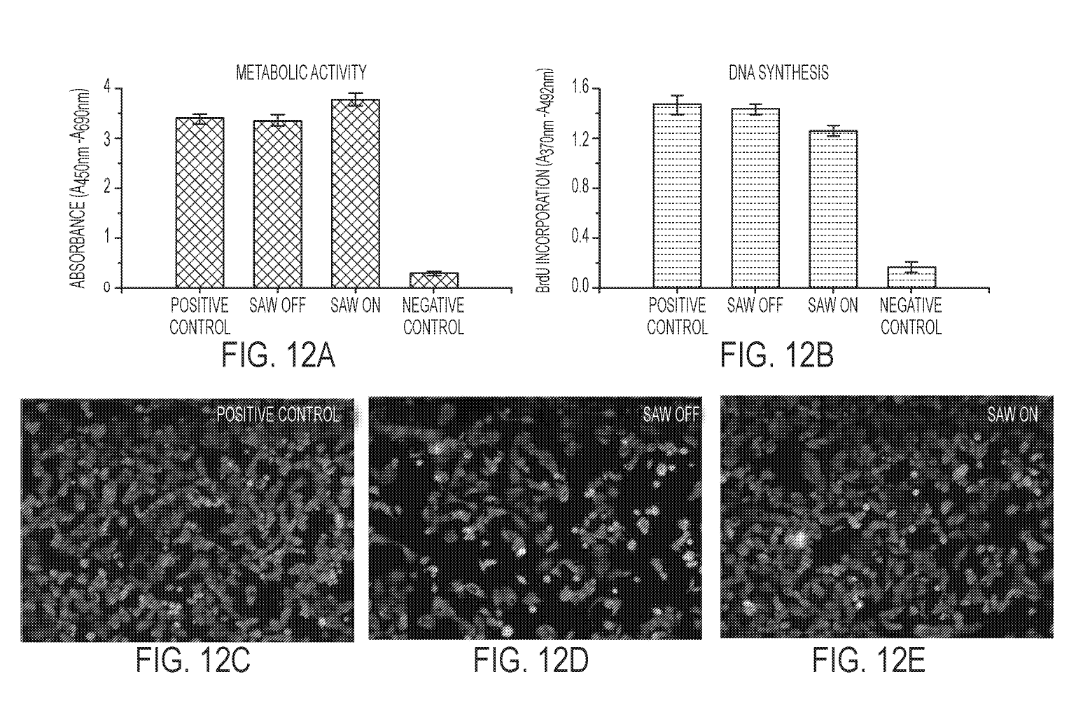

Viability and proliferation assays were conducted to verify that the device is safe to biological cells for the duration of the experiment. The WST-1 cell viability test (Roche, Nutley, N.J.) and the BrdU Cell Proliferation ELISA (Roche, Nutley, N.J.) were used to test the cells' viability and proliferation, respectively. MCF-7 cells were delivered into the device at a flow rate of 2 μL/min under an input power of 25 dBm (˜2 W/cm²). Cell tests were then carried out immediately after collecting them from the outlet. Cells that were not processed in the device and those flowing through the device with SAW turned off were also tested as control experiments. Cell viability and proliferation were examined by measuring cell metabolic activity and DNA synthesis, as shown in FIG. 12, Panels A and B. Direct staining of cell viability, as shown in FIG. 12, Panels C, D, and E, indicates that no significant damage was found in the physiological properties of the processed cells as a consequence of the taSSAW experiment.

A taSSAW-based, label-free, cell-separation device was developed that can achieve high separation efficiency, high sensitivity, and high biocompatibility simultaneously compared to existing methods. By aligning the SSAW-induced pressure nodal lines at an inclined angle to the flow direction, the micro-channel was covered with a series of pressure nodal lines, a design feature that significantly improved the cell-separation efficiency and sensitivity over previous acoustic cell separators.

Acoustic radiation force Fᵣ is a periodic function of space (as shown in Eqs. S2 and S4), rather than a constant, so that if the acoustic radiation force is orthogonal to the fluid direction, particles in different sides of the nodal lines will experience forces with opposite signs, and they will eventually converge to a nodal line, leading to no separation. However, in the tilted-angle case, smaller particles will experience smaller acoustic forces and be pushed by the flow field to cross multiple nodal lines, leading to separation from larger particles. If all particles are subjected to large acoustic forces, they will converge to a single nodal line even in the tilted-angle case. Thus in the case of 0=90° (acoustic force is orthogonal to fluid field), there will be no separation if the channel is long enough for particles to converge. On the other hand, if 0=0° and the acoustic force is in the same direction with the fluid field, there is no lateral motion of particles so that there is no separation either. Therefore, an optimal separation is achieved for θ<θ<90°, as clearly shown by the simulation results in FIG. 6.

Using the taSSAW approach, it was possible to separate 10 μm and 2 μm polystyrene beads with a 99% separation efficiency (FIG. 1, Panels B and C). With the taSSAW design, separation of particles with small size differences was also achieved: separation of particles with diameter of 7.3 from 9.9 μm diameter particles with a 97% separation efficiency.

The successful separation of three different particles of 4 μm, 10 μm, and 15 μm diameter in one step demonstrates the potential of the method presented here to simultaneously separate multiple components of a sample. Such a capability could be applied to achieve separation of plasma, platelets, RBCs, and WBCs in human blood in a single step. The ability to distinguish cells/particles with different mechanical properties (such as compressibility) offers new avenues for cell separation for disease diagnostics (e.g., distinguishing between RBCs invaded by malaria-inducing Plasmodium falciparum versus Plasmodium vivax parasites) and for elucidating the underlying mechanistic processes.

This method is able to separate cancer cells from normal WBCs, thereby providing a unique approach for the isolation and detection of circulating tumor cells and addressing an important challenge in cancer biology. Under the current experimental conditions, a recovery rate of 71% and a purity of 84% was achieved, as shown in FIG. 10, Panel H. Further improvements to the device performance could be made by optimizing device-operating parameters (such as channel dimensions, input power, and inclination angle) and/or hardware (such as electronics and piezoelectric substrate). The taSSAW technology, has the potential to address one of the significant challenges in cancer research and diagnostics: isolating a small number of leukemia cells from normal leukocytes, which may have similar size but different compressibility (33). The simulation results (FIG. 3), for the power and inclination angle of 30 dBm and 8 degrees, respectively, shows that a small difference in compressibility (from the normal value of 3.99×10⁻¹⁰ Pa⁻¹ to 3.75×10⁻¹⁰ Pa⁻¹) is sufficient for effective separation of these softer cells from normal leukocytes. These results show that the taSSAW technology is very sensitive to cell compressibility, which provides an alternative route for cell separation. This route would be suitable for applications involving cells with similar size, but different compressibility.

Furthermore, the acoustic power intensity and frequency used in the taSSAW separation methods are in a similar range as those used in ultrasonic imaging, which has proven to be extremely safe for health monitoring, even during pregnancy. At these power intensities and frequencies, acoustic waves tend to be gentle to biological cells. As a result, cell integrity may be preserved during the acoustic separation process. The biocompatible nature of the approach was validated by cell viability and proliferation assays, in which no significant damage was found in the physiological properties of the processed cells (FIG. 12, Panels A-E).

To maintain high separation sensitivity and efficiency, the taSSAW device was found to work well at a throughput of ˜2 μL/min for cells (10,000-20,000 cells/min) and ·20 μL/min for polystyrene beads. This throughput might be sufficient for many medical diagnostic systems. Other optimal settings in cell-separation throughput for cancer detection in clinical practice (e.g., isolation of a small number of circulating tumor cells in a large population of cells) or therapeutics applications (e.g., transfusion) can be identified using the guidance provided herein.

Finally, the taSSAW-based device is compact and inexpensive. SAW devices have been used extensively in many microelectronics industries, including the cell phone industry. This industrial base for SAW devices and associated accessories has dramatically lowered the cost and improved the reliability of these components. The simple design, low cost, and standard fabrication process of the device outlined here allows for easy integration with other lab-on-a-chip technologies and small RF power supplies to further develop a fully integrated cell separation and analysis system. With its advantages in biocompatibility, efficiency, sensitivity, and simplicity, the taSSAW-based method presented here thus provides new avenues for furthering acoustic tweezers technologies and for many biological studies and clinical applications.

### Materials and Methods

The fabrication of the taS SAW microfluidic device involves three major steps: (1) the fabrication of inter-digital transducers (IDTs) on a lithium niobate (LiNbO3) piezoelectric substrate, (2) the fabrication of PDMS microchannel, and (3) bonding of the PDMS channel onto the LiNbO3 piezoelectric substrate with IDTs. The fabrication of IDTs consists of photolithography, metal deposition, and lift-off process. One layer of photoresist (SPR3012, MicroChem, Newton, Mass.) was first spin-coated on a Y+128° X-propagation LiNbO3 wafer, patterned with a UV light source, and then developed in a photoresist developer (MF CD-26, Microposit, The Dow Chemical Company, Midland, Mich.). Plasma surface cleaning (Metroline M4L Plasma Etcher, PVA TEPLA, Corona, Calif.) was applied before a double-layer metal (Cr/Au, 50 Å/500 Å) was deposited on the LiNbO3 wafer using an e-beam evaporator (Semicore Corp, Livermore, Calif.). Subsequently a lift-off process was followed to remove the photoresist and to form the IDTs. The PDMS microchannels were fabricated using standard photolithography and mold-replica techniques. A photolithography was used to pattern the silicon substrate using photoresist (SU-8, MicroChem, Newton, Mass.), followed by a post-exposure baking at 150° C. for 30 min. SylgardTM 184 Silicone Elastomer Curing Agent (Dow Corning, Midland, Mich.) and SylgardTM 184 Silicone Elastomer Base were mixed at 1:10 weight ratio, cast on top of the silicon mold, and cured at 65° C. for 30 min. Finally, the IDT substrate and the PDMS microchannel were both treated with oxygen plasma and bonded together, and connected with polyethylene tubing (inner diameter: 280 μm). During the experiments, a taSSAW microfluidic device was mounted on the stage of an inverted microscope (Nikon TE2000U). IDTs were electrically connected to a function generator (Agilent E4422B, Santa Clara, Calif.) through an amplifier (AR 250A100, Bothell, Wash.). PDMS channel inlet and outlet were connected to syringes driven by Nemesys syringe pumps (Cetoni GmbH, Korbussen, Germany). A CCD camera (CoolSNAP HQ2, Photometrics, Tucson, Ariz.) and a high-speed camera (CoolSNAP HQ2, Photometrics, Tucson, Ariz.) were connected to the microscope to record the separation process.

### Cell Preparation and Cell Staining

Fresh human whole blood with acid citrate dextrose (ACD) as an anticoagulant was purchased from Zen-bio, Durham, N.C. In order to lyse the RBCs, 1 ml of whole blood was mixed with 10 ml of 1× RBC lysis buffer (eBioscience, San Diego, Calif.) and incubated for 10-15 min at room temperature. After centrifugation at 400 × g for 5 min to remove the supernatant, white blood cells (WBCs) were resuspended in 1 ml of 4% paraformaldehyde in PBS (Santa Cruz Biotechnology, Dallas, Tex.) and fixed at room temperature for 10 min. After cell counting with a hemacytometer to determine the cell concentration, the fixed WBCs were centrifuged and re-suspended in PBS before use.

MCF-7 breast cancer cells were cultured in Dulbecco's modified Eagle medium, (DMEM)-F12 medium (Life Technologies, Grand Island, N.Y.), with 10% fetal bovine serum (Life Technologies), penicillin (100 U/ml), and 100 μg/ml streptomycin (Mediatech, Manassa, Va.) to about 90% confluence. Then the MCF-7 cells were trypsinized (Trypsin+0.05% EDTA, Life Technologies, Grand Island, N.Y.), centrifuged, and resuspended in 4% paraformaldehyde in PBS (Santa Cruz Biotechnology, Dallas, Tex.) for cell fixation. After cell counting with a hemacytometer to determine the cell concentration, the fixed MCF-7 cells were centrifuged and resuspended in PBS before use.

After the MCF-7 cells were spiked into the WBCs at desired ratios, these cells were centrifuged and re-suspended in 500 μl of PBS with 5 μl of 300 nM DAPI in PBS (Life Technologies, Grand Island, N.Y.) added for staining of cell nuclei at room temperature for 10 min. Then the cells were washed with PBS, centrifuged, and re-suspended in 200 μl of PBS, after which 5 μl of FITC-conjugated anti-CD45 antibody (Life Technologies, Grand Island, N.Y.) was added to stain the surface of WBCs for 10 min at room temperature. After another wash with PBS and centrifugation, the cells were again re-suspended in 200 μl of PBS with 5 μl of Phycoerythrin (PE)-conjugated anti-EpCAM antibody (eBioscience, San Diego, Calif.) added to stain the surface of MCF-7 cells for 10 min at room temperature. After all the staining steps, the cells were washed and suspended in PBS.

### Cell Viability and Proliferation Tests

Post-separation evaluations of viability and proliferation on taSSAW-treated live MCF-7 cells were conducted. Live MFC-7 cells suspended in fresh medium were introduced into the microchannel and collected at the outlet either with SAW applied (“SAW on” group) or without SAW (“SAW off” group). Live MCF-7 cells without any treatment were used as positive control.

The viability of MCF-7 cells was first measured using live/dead cell staining. MCF-7 cells in each group were counted with a hemacytometer and seeded into 35 mm tissue culture dishes at seeding density of 4×10⁵ cells/dish. After culture for 24 h, the cell monolayers were stained with 500 ng/ml of Calcein AM (live cell staining) (Life Technologies, Grand Island, N.Y.) and 1 μM of SYTOX ® Orange (dead cell staining) (Life Technologies, Grand Island, N.Y.) to evaluate the number of apoptotic cells in each group under epifluorescence imaging.

For cell viability and proliferation assays, cells in each group were first counted with a hemacytometer and diluted with fresh medium to 4×10⁵ cells/ml, and then seeded in a Costar 96-well black clear-bottom plate (Corning Life Sciences, Tewksbury, Mass.) with cell seeding density of 2×10⁴ cells/well within a 100 μl culture medium. For each group, 5 repeat wells were seeded. MCF-7 cells were then cultured in an incubator at 37° C. and 5% CO₂ for 20 h when 10 μl/well BrdU labeling solution (Roche Applied Science, Indianapolis, Ind.) was added into each well. After 2 h of incubation, 10 μl/well WST-1 (Roche Applied Science, Indianapolis, Ind.) was added into each well. After another 2 h of incubation, the absorbance of each well at 450 nm and 690 nm (reference wavelength) using a microplate reader (BioTek, Winooski, Vt.) was measured to assess cell viability. Cell proliferation was then measured by characterizing BrdU incorporation using Cell Proliferation ELISA, BrdU (colorimetric) (Roche Applied Science, Indianapolis, Ind.). The absorbance of each well at 370 nm and 492 nm (reference wavelength) was measured after finishing the standard ELISA procedure.

### Patient Blood Processing and Image Acquisition

Within 24 h of collection, blood cells were first lysed using RBC lysis buffer for 5 min. to remove the majority of erythrocytes. WBCs were collected by centrifuging the blood solution at 800×g and resuspended in the same volume as the whole blood in 1× PBS solution with 0.1% PF-68. Then the sample was run through the taSSAW device at a flow rate of 1.2 mL/h. The sample was then taken from the collection outlet and concentrated into one 1.7 mL centrifuge tube for immunofluorescence staining. A standard immunofluorescence staining protocol was followed. Cells were first fixed and permeabilized using 4% paraformaldehyde and 0.1% Triton X-100, and then were incubated with fluorescence dye-labeled primary antibodies and secondary antibodies. All the bright-field and fluorescence images were taken using an inverted Nikon microscope and a charged coupled-device (CCD) camera.

### Immunofluorescence Staining and Image Acquisition

For immunofluorescence staining, cells were first fixed using 4% paraformaldehyde (ChemCruz, USA) for 10 min at room temperature and permealized by 0.1% Triton X-100 (Sigma, USA) in PBS solution for 10 min at room temperature. Cells were then blocked by 200 uL of 3% BSA in PBS solution (Life Technologies, USA) for 30 min. After blocking non-specific binding sites, 5 μL FITC labelled mouse anti-cytokeratin (CK) 8, 18 (Abcam, USA), 1 μL rabbit anti-ER (CellSignal, USA), and 5 μL Cy5 labelled mouse anti-CD45 (eBioscience, USA) were added into the 3% BSA solution and incubated at 4° C. overnight. After overnight incubation, cells were washed with PBS and stained with 5 μL PE labelled anti-rabbit IgG (Life Technologies, USA) for 2 h at room temperature. Before transporting cells to a chamber slide for observation, DAPI was added to the cell solution for nuclei staining.

Both bright field and fluorescence images were obtained using an inverted epifluorescence microscope (Ti-U Eclipse, Nikon, Japan) and a charge-coupled device (CCD) camera (CoolSNAP HQ², Photometrics, USA). 4× (0.10 NA), 10× (0.30 NA), 20× (0.45 NA), and 60X (0.85 NA) objectives were used in the experiment. For CTC counting experiment, the whole chamber slide was scanned manually with a 20× objective. For CTC imaging under the 60× objective, the cell samples were transferred to a customized PDMS chamber with a NO 1. glass slide as the bottom. All the microscopy images were processed and analysed using ImageJ (NIH, USA).

### Example 2

### Separation of Low-Abundance Cancer Cells from Human Blood Using Surface Acoustic Waves

Cancerous cells presenting in the blood flow are called circulating tumor cells (CTCs). CTCs carry important information related to disease progression and prognosis. It opens up the opportunity to examine and study cancer progress with a more non-invasive manner (blood drawing). However, it is extremely challenging to isolate CTCs from blood sample due to the scarcity of CTCs (1-100 cancer cells in one mL of blood).

This invention presents, for the first time, the separation of human cancer cells from human white blood cells using surface acoustic wave technologies (FIG. 13). It is also the first report of acoustic-based separation of rare cancer cells from human blood (<1000 cancer cells per ml blood) which maintains high cell viability and integrity. The ability to preserve cell viability and integrity is significant for downstream analysis. With its ability to achieve high-efficiency, high-biocompatibility, and high-throughput simultaneously, the invention will be invaluable in cancer research and clinical treatment. It enables label-free, non-invasive isolation of rare cancer cells from patients' blood and allows clinicians to monitor disease progress, predict therapy outcome, and find out genetic mutations of cancer patients.

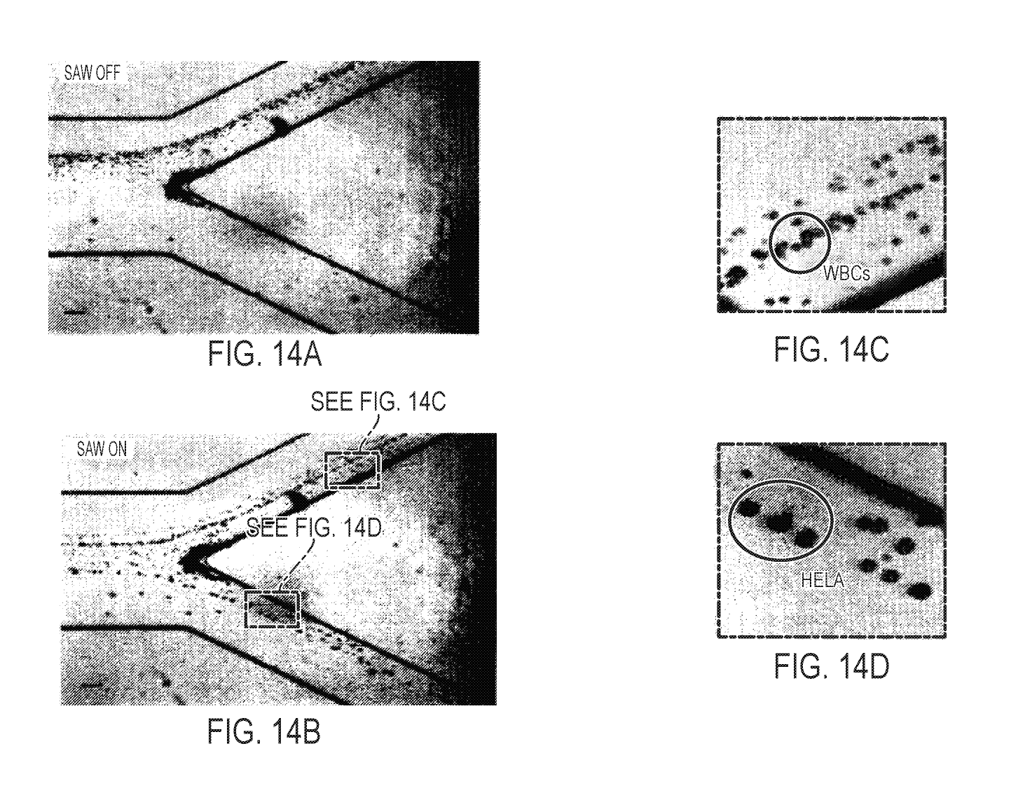

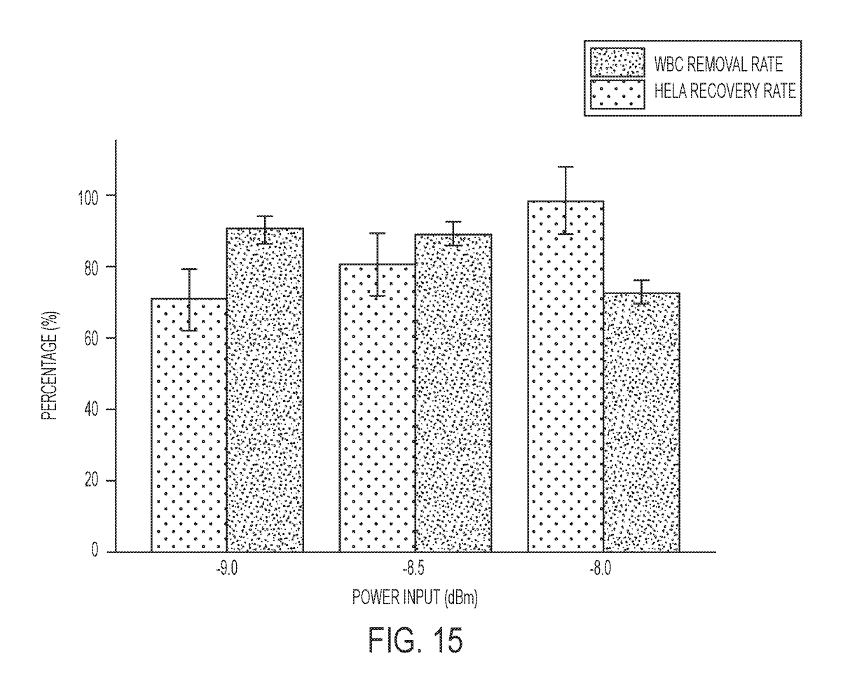

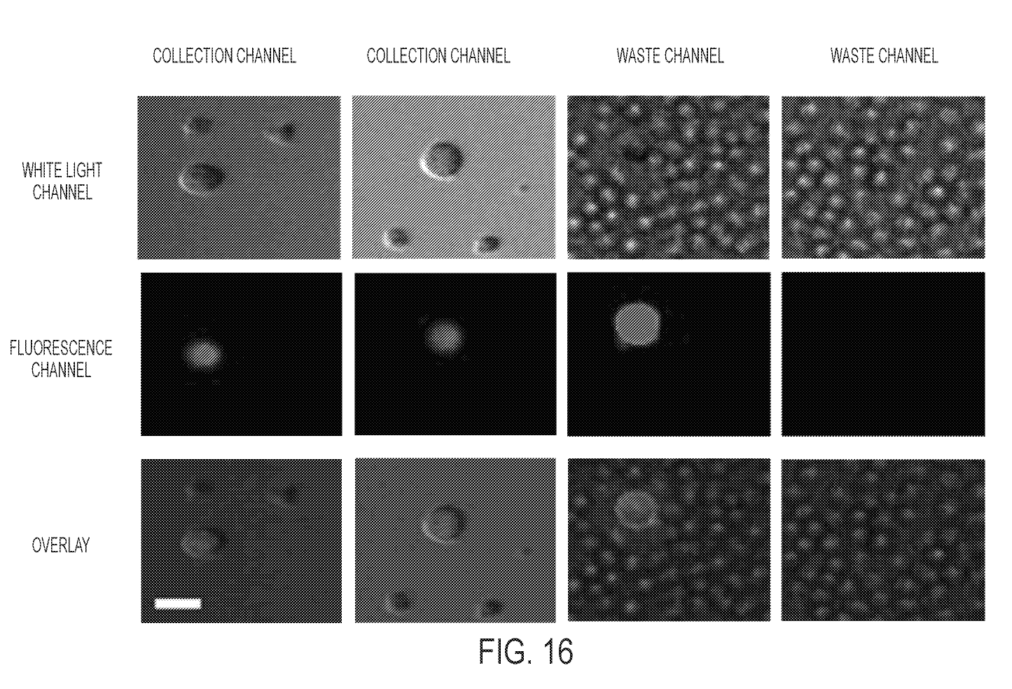

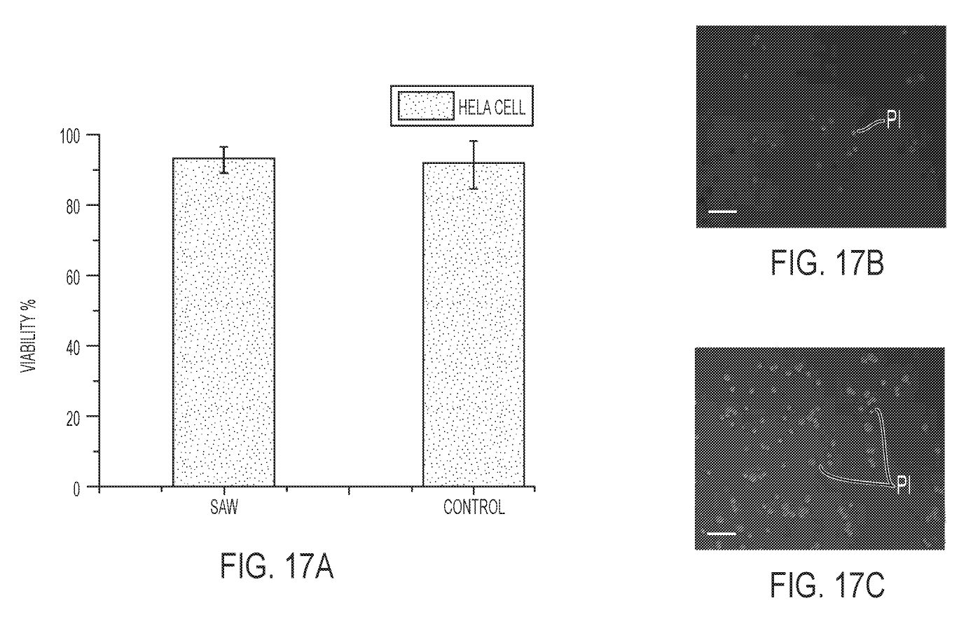

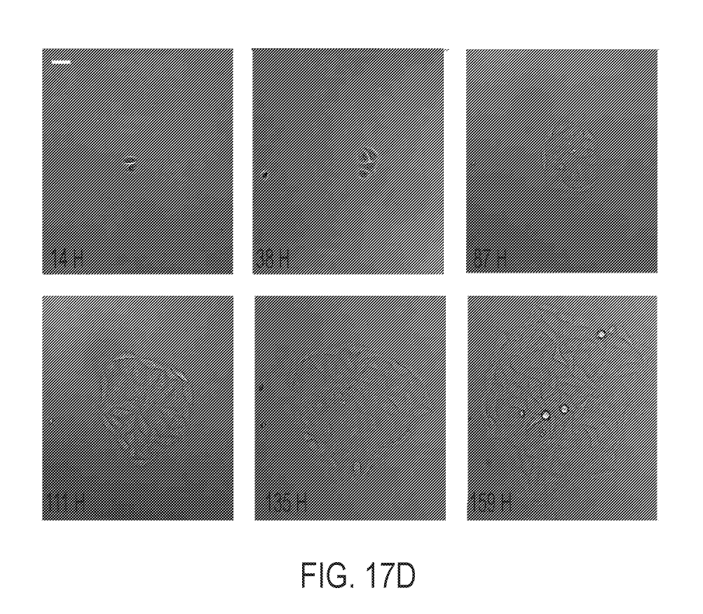

The separation of cancer cells (HeLa) from human white blood cells under a surface acoustic wave field (FIG. 14, Panels A-D) was demonstrated. The dependence of separation performance on input power was also studied (FIG. 15). Then 100-1000 Hela cells were mixed with 1 mL of human white blood cells; the resulted mixture were separated by applying a standing surface acoustic wave field. The results showed that >80% separation efficiency can be achieved for the rare cell sample (FIG. 16, and Table 1). The current single device is able to process 1.2 mL of blood per hour (2000-3000 cells/s), and cells are still viable after separation (FIG. 17, Panels A-D). The cells were loaded into the taSSAW device at a flow rate of 1.2 mL/h for high-throughput separation. The power input was set at 37-38 dBm, and the input frequency was 19.573 MHz. Cells, from both the collection and waste outlets, were collected into two different petri dishes with depth and height of 35 mm and 10 mm, respectively. The number of fluorescent cells in both collection and waste outlets was counted, and the recovery rate was obtained by dividing the number of cells in the collection channel by the number of total cells from both outlets.

After successfully demonstrating rare cell separation with MCF-7 and HeLa cells, the high-throughput taSSAW device was tested with other cancer cell lines. Since the physical properties of cells from real cancer patients are unknown a priori, it is important to test the tolerance of the device with different cell lines. For this purpose, melanoma and prostate cancer cell models, UACC903M-GFP cells and LnCAP cells, respectively, were used. Unlike the previous separation for MCF-7 and HeLa cells in which the separation parameters using abundant cell samples were optimized, rare cell concentrations of UACC903M-GFP cells and LnCAP cells were directly tested using the same operating parameters to separate the MCF-7 and the HeLa cells. As shown in Table 1, a recovery rate greater than 85% for these cell lines was also obtained, indicating the robustness of the device for separating different cancer cell lines.

TABLE 1
Rare cell separation with multiple-spiked cancer cell lines.

| | | No. of | | |
|---|---|---|---|---|
| | Cells ratio | cancer cells | No. of | |
| | (Cancer | (collection | Cancer cells | Recovery |
| Cell line | cells/WBCs) | outlet) | (waste outlet) | rate |
| MCF-7 | 1:40000 | 121 | 20 | 86% |
| MCF-7 | 1:40000 | 90 | 11 | 89% |
| MCF-7 | 1:40000 | 52 | 2 | 96% |
| HeLa | 1:6000 | 907 | 165 | 85% |
| HeLa | 1:100000 | 49 | 10 | 83% |
| HeLa | 1:100000 | 53 | 15 | 78% |
| HeLa | 1:40000 | 131 | 7 | 95% |
| UACC903M-GFP | 1:100000 | 64 | 12 | 84% |
| UACC903M-GFP | 1:100000 | 36 | 7 | 84% |
| UACC903M-GFP | 1:100000 | 28 | 5 | 85% |
| LnCAP | 1:20000 | 111 | 12 | 90% |

### Example 3

### Separation of Cells and/or Particles Using Segmented Interdigital Transducers (S-IDTs)

### Microfluidic Device

As shown in FIG. 18, Panel A, to generate standing surface acoustic waves (SSAW), a pair of interdigital transducers (IDT) is deposited on a piezoelectric substrate (LiNbO₃). A polydimethylsiloxane (PDMS) based microfluidic channel is bonded onto the substrate with three inlets (two sheath flows and one sample flow) and two outlets. The size of the device is slightly larger than a penny.

Instead of using the regular parallel IDTs, a pair of segmented IDTs (S-IDT) were used. As shown in the inset of FIG. 18, Panel A, a S-IDT consists of many small sections of parallel IDTs. Each section has a consistent displacement from the previous one in the lateral direction. The function of a S-IDT is to generate many discontinued, independent SSAW fields in the fluidic channel along the flow direction. Each field has a certain phase shift from the previous one as shown in FIG. 18, Panel B. As the particles/cells enter the phase-shifting SSAW fields, they will experience acoustic radiation forces and follow the pressure nodes (minimum pressure). The particles thus have a zigzag movement because of the continuous phase shift of the SSAW fields. FIG. 18, Panel C shows the stacked images of particle trajectories. When SSAW is off, the particles follow the streamline with a straight trajectory. When SSAW is on, particles have a zigzag trajectory at a low flow rate. As the flow rate increases, the trajectory becomes smooth with a displacement in the lateral direction.

### Separation Mechanism

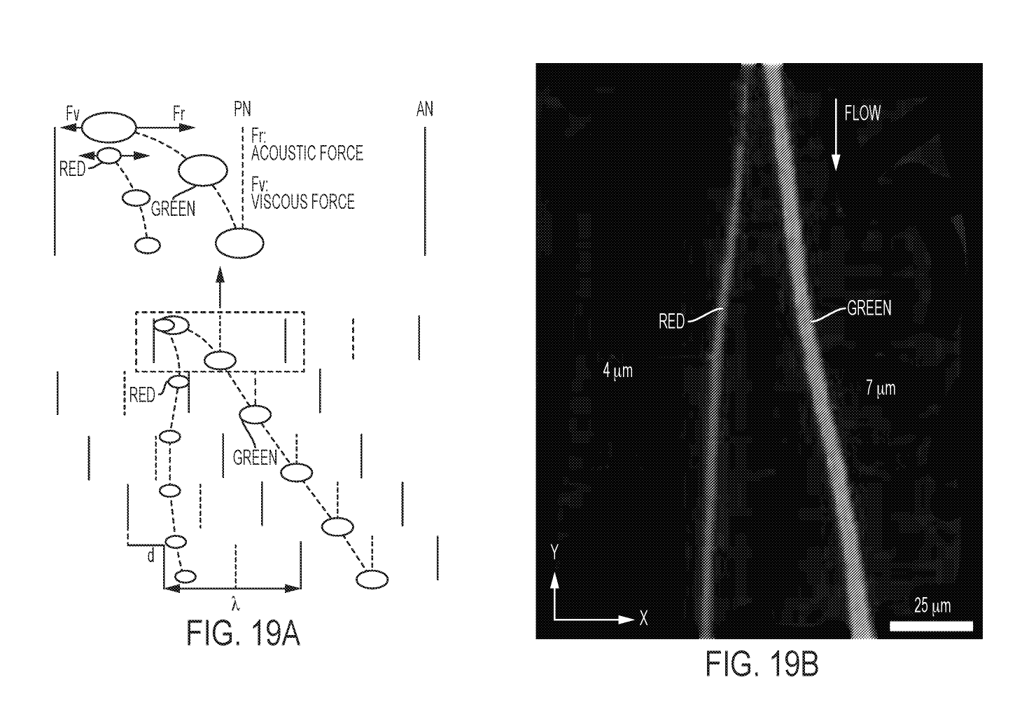

When two particles/cells with difference sizes (or mechanical properties) go through the phase-shifting SSAW fields, they will follow the trajectories as shown in FIG. 19, Panel A. At the beginning, both of the particles experience acoustic radiation forces and move in the same direction. The difference in the radiation force intensities results in their different lateral displacements, By optimizing the flow rate and acoustic power, two particles will then go into different pressure nodes (PN) with a pressure antinode (AN) between them. By periodically repeating this process, the small particles and big particles will have a significant distance for collection from different outlets. FIG. 19, Panel B shows the 4 μm. and 7 μm particles flow through phase-shifting SSAW fields. The 4 μm particles move towards negative x direction, while the 7 μm particles move towards positive x direction. As a result, at least two types of particles can be separated along the x axis (lateral direction).

A major difference and advantage of this invention over other SSAW-based particle/cell separation technologies lie in its high-resolution identification of different particles/cells. The conventional SSAW based particle/cell separation technologies employ regular IDTs³⁴, which push all the particles/cells towards the same pressures node. The particles/cells are separated because they experience different intensities of acoustic radiation forces. Once the particles have very similar properties, they are difficult to be separated. However, in the phase-shifting SSAW fields, particles have a chance to experience acoustic radiation forces from different directions and move to different pressure nodes. Therefore, the separation distance can be increased with more S-IDT segments, and consequently the separation resolution can be significantly increased.

### Example 4

### Probing CTCs from Breast Cancer Patient Blood Samples

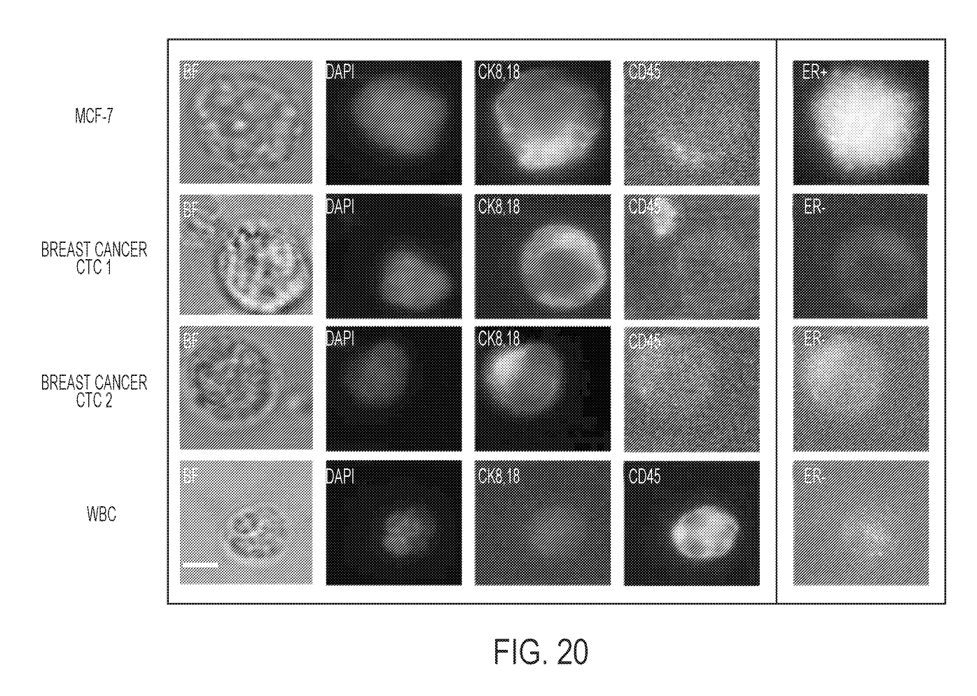

As a practical demonstration, the microfluidic device, described herein, was tested with blood samples obtained from three patients with metastatic breast cancer. Immunofluorescent staining of cytokeratin (CK) and pan-leukocyte marker CD45 was used to first determine the identity of the cells using conventional detection methods. Cells were identified as CTCs if the immunofluorescent pattern is DAPI+/CK8,18+/CD45−, otherwise cells were identified as WBCs. Based on this immunostaining detection criteria, 59 and 8 CTCs found in the first two patients, respectively, in 2 mL of blood samples from each patient. Both of these patients had CTCs detected by the Veridex assay on previous occasions. The third patient had only 1 CTC after screening 6 mL of blood. Typical fluorescent images are shown in FIG. 20. WBCs can be distinguished from the CTCs as they showed an immunostaining pattern of DAPI+/CK8,18−/CD45+. Using immunofluorescence, one could also check the expression of certain protein markers in patients' CTCs after taSSAW-based separation. In this case, the expression of ER in the patients' CTCs was examined. As shown in FIG. 20, the MCF-7 cell line was used as a positive control for ER. Compared to the fluorescent signal from the ER in the MCF-7 cells, all the patient samples were considered negative for ER. All three patients had initial diagnosis of ER+ breast cancer. The first two patients (with CTC counts of 59 and 8 respectively) had been heavily pretreated with multiple lines of endocrine therapy as well as chemotherapy and had chemotherapy refractory disease. In the first patient immunohistochemical staining of a biopsy of a metastatic site (bone) showed persistent ER positivity and PR positivity at 5%; the second patient had biopsy of her metastatic tumor in the pelvis and this was ER positive at 100% and PR positive at 1%. Loss of ER positivity in the CTCs from patients with initial diagnosis of ER positive breast cancer has been previously reported and could be a reflection of tumor heterogeneity (38). The third patient had a low (one single CTC) CTC count, which is consistent with the fact that the blood was drawn within 2 months after initiating a new line of endocrine therapy to which she responded; this was ascertained by clinical examination and imaging (CT scans).

### REFERENCES

1. Eisenstein M (2006) Cell sorting: Divide and conquer. Nature 441:1179-1185.
2. Nagrath S, et al. (2007) Isolation of rare circulating tumour cells in cancer patients by microchip technology. Nature 450:1235-1239.
3. Neu\check{z}i P, Giselbrecht S, Lange K, Huang T J, Manz A (2012) Revisiting lab-on-a-chip technology for drug discovery. Nat Rev Drug Discov 11:620-632.
4. Kotz K T, et al. (2010) Clinical microfluidics for neutrophil genomics and proteomics. Nat Med 16:1042-1142.
5. Bosio A, et al. (2009) Isolation and enrichment of stem cells. Adv Biochem Eng Biotechnol 114:23-72.
6. Gossett D R, et al. (2010) Label-free cell separation and sorting in microfluidic systems. Anal Bioanal Chem 397:3249-3267.
7. Lenshof A, Laurell T (2010) Continuous separation of cells and particles in microfluidic systems. Chem Soc Rev 39:1203-1217.
8. Bose S, et al. (2013) Affinity flow fractionation of cells via transient interactions with asymmetric molecular patterns. Sci Rep 3:2329.
9. Grover W H, et al. (2011) Measuring single-cell density. Proc Natl Acad Sci USA 108:10992-10996.
10. Du E, et al. (2013) Electric impedance microflow cytometry for characterization of cell disease states. Lab Chip 13:3903-3909.
11. Bao G, Suresh S (2003) Cell and molecular mechanics of biological materials. Nat Mater, 2:715-725.
12. Suresh S (2007) Biomechanics and biophysics of cancer cells. Acta Biomater 3:413-438.
13. Wei H, et al. (2011) Particle sorting using a porous membrane in a microfluidic device. Lab Chip 11:238-245.
14. Schirhagl R, Fuereder I, Hall E W, Medeiros B C, Zare R N (2011) Microfluidic purification and analysis of hematopoietic stem cells from bone marrow. Lab Chip 11:3130-3135.
15. Huang L R, Cox E C, Austin R H, Sturm J C (2004) Continuous particle separation through deterministic lateral displacement. Science 304:987-990.
16. Zeming K K, Ranjan S, Zhang Y (2013) Rotational separation of non-spherical bioparticles using I-shaped pillar arrays in a microfluidic device. Nat Commun 4:1625.
17. Davis J A, et al. (2006) Deterministic hydrodynamics: taking blood apart. Proc Natl Acad Sci USA 103:14779-14784.
18. Carlo D D, Irimia D, Tompkins R, Toner M (2007) Continuous inertial focusing, ordering, and separation of particles in microchannels. Proc Natl Acad Sci USA 104:18892.
19. Carlo D D (2009) Inertial microfluidics. Lab Chip 9:3038-3046.
20. Guan G, et al. (2013) Spiral microchannel with rectangular and trapezoidal cross-sections for size based particle separation. Sci Rep 3:1475.
21. Huang S B, et al. (2013) High-purity and label-free isolation of circulating tumor cells (CTCs) in a microfluidic platform by using optically-induced-dielectrophoretic (ODEP) force. Lab Chip 13:1371-1383.
22. Sun J, et al. (2012) Simultaneous On-Chip DC dielectrophoretic cell separation and quantitative separation performance characterization. Anal Chem 84:2017-2024.
23. Petersson F, Nilsson A, Holm C, Jonsson H, Laurell T (2005) Continuous separation of lipid particles from erythrocytes by means of laminar flow and acoustic standing wave forces. Lab Chip 5:20-22.
24. Lenshof A, Magnusson C, Laurell T (2012) Acoustofluidics 8: Applications of acoustophoresis in continuous flow microsystems. Lab Chip 12:1210-1223.
25. Ding X, et al. (2012) On-chip manipulation of single microparticles, cells, and organisms using surface acoustic waves. Proc Natl Acad Sci USA 109:11105-11109.
26. Nam J, Lim H, Kim D, Shin S (2011) Separation of platelets from whole blood using standing surface acoustic waves in a microchannel. Lab Chip, 11:3361-3364.
27. Shi J, et al. (2009) Continuous particle separation in a microfluidic channel via standing surface acoustic waves (SSAW). Lab Chip 9:3354-3359.
28. Shi J, Mao X, Ahmed D, Colletti A, Huang T J (2008) Focusing microparticles in a microfluidic channel with standing surface acoustic waves (SSAW). Lab Chip 8:221-223.
29. Shi J, Ahmed D, Mao X, Lin S S, Huang T J (2009) Acoustic tweezers: patterning cells and microparticles using standing surface acoustic waves (SSAW). Lab Chip 9:2890-2895.
30. Ding X, et al. (2012) Standing surface acoustic wave (SSAW) based multichannel cell sorting. Lab Chip 12: 4228-4231.
31. Gallo J A, Draper D O, Brody L T, Fellingham G W (2004) A comparison of human muscle temperature increases during 3-MHz continuous and pulsed ultrasound with equivalent temporal average intensities. J Orthop Sports Phys Ther 34:395-401.
32. Ding X, et al. (2013) Surface acoustic wave microfluidics. Lab Chip, 13:3626-3649.
33. Zhou Z, Hui T, Tang B, Ngan A (2014) Accurate measurement of stiffness of leukemia cells and leukocytes using an optical trap by a rate-jump method. RSC Adv 4:8453-8460.
34. Jinjie Shi, Hua Huang, Zak Stratton, Aitan Lawit, Yiping Huang and Tony Jun Huang, Continuous Particle Separation in a Microfluidic Channel via Standing Surface Acoustic Waves (SSAW), Lab on a Chip, Vol. 9, pp. 3354-3359,2009.
35. Hartono D, Liu Y, Tan P L, Then X Y, Yung L Y, Lim KM. On-chip measurements of cell compressibility via acoustic radiation. Lab Chip. 2011 Dec. 7; 11(23):4072-80.
36. Michel Godin, Andrea K. Bryan, Thomas P. Burg, Ken Babcock and Scott R. Manalis. Measuring the mass, density, and size of particles and cells using a suspended microchannel resonator. Appl. Phys. Lett. 91,123121 (2007).
37. GE Healthcare. Cell integrity assays. High-content analysis of essential cell integrity and toxicity parameters using the IN Cell Analysis System. https://www.gelifesciences.com/gehcls_images/GELS/Related%20Content/Files/1314774443 672/litdoc28408716AA_20110831103235.pdf
38. Babayan A, et al. (2013) Heterogeneity of estrogen receptor expression in circulating tumor cells form metastatic breast cancer patients. PLos One 8(9): e75038.

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one or all of the group members are present in, employed in or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method for separating cancer cells from a mixed population of cells, comprising
flowing a sample containing a mixed population of cells through a channel, wherein the mixed population of cells includes cancer cells and non-cancer cells, subjecting the sample to a surface acoustic wave (SAW), causing the sample to separate into two flowing streams of sample, wherein the first flowing stream of sample has cancer cells and the second flowing stream has non-cancer cells, wherein the cancer cells comprise less than 10% of the total mixed population of cells.

2.-3. (canceled)

4. The method of claim 1, wherein the cancer cells comprise less than 5% of the total mixed population of cells.

5.-7. (canceled)

8. The method of claim 1, wherein the cancer cells are circulating tumor cells (CTCs).

9. The method of claim 8, wherein the CTCs comprise 1-100 cancer cells in one mL of blood and optionally, wherein the CTCs comprise 1-10 cancer cells in 7.5 mL of blood.

10.-11. (canceled)

12. The method of claim 1, wherein greater than 80% separation efficiency of the cancer cells from the mixed cell population is achieved.

13.-15. (canceled)

16. The method of claim 1, wherein at least 1 mL of sample per hour is processed and optionally, wherein a flow rate of the fluid is 1-500 μL/min.

17. The method of claim 1, wherein the surface acoustic wave is generated by at least two interdigital transducers (IDT).

18. The method of claim 1, wherein the surface acoustic wave is generated by at least two segmented interdigital transducers (S-IDT).

19.-36. (canceled)

37. An apparatus for sorting cells from a mixed population of cells, comprising:
a surface, a channel on the surface, the channel having an inlet end and an outlet end, wherein the inlet end comprises at least one inlet and the outlet end comprises at least two outlets; the channel having a direction from the inlet end to the outlet end;
a first surface acoustic wave (SAW) generator, wherein the SAW generator is a segmented interdigital transducer (S-IDT);
a second SAW generator, wherein the second SAW generator is a S-IDT;
the first and second SAW generators being operably configured on the surface, and on opposing sides of the channel to generate a SAW within the channel between the inlet end and outlet end of the channel and having a SAW direction.

38. The apparatus of claim 37, wherein the SAW direction is disposed at a 10-45 degree angle to the channel direction.

39. An apparatus for sorting cells from a mixed population of cells, comprising:
a surface, a channel on the surface, the channel having an inlet end and an outlet end, wherein the inlet end comprises at least one inlet and the outlet end comprises at least two outlets; the channel having a direction from the inlet end to the outlet end; wherein, the surface acoustic wave direction is disposed at an angle to the channel direction, and
first and second SAW generators operably configured on the surface, and on opposing sides of the channel to generate a SAW within the channel between the inlet end and outlet end of the channel and having a SAW direction, wherein the SAW direction is disposed at a 10-25 degree angle.

40. The apparatus of claim 39, wherein the SAW direction is disposed at a 15 degree angle.

41. The apparatus of claim 37, wherein: the surface is a piezoelectric substrate; and the first and second SAW generators each comprise electrodes supported by the surface.

42. The apparatus of claim 37, wherein the apparatus is a microfluidic device; the channel is a microchannel; and the microchannel has at least one cross-sectional dimension less than 1 mm.

43. The apparatus of claim 37, wherein the SAW direction is at a non-oblique angle to the channel direction.

44. The apparatus of claim 37, wherein the SAW direction is at an oblique angle to the channel direction.

45. (canceled)

46. The apparatus of claim 37, wherein the SAW direction is at an angle ranging from 10-15 degrees to the channel direction.

47. The apparatus of claim 37, wherein the SAW direction is at an angle ranging from 15-30 degrees to the channel direction.

48. The apparatus of claim 37, wherein the SAW direction is at an angle ranging from 30-45 degrees to the channel direction.

49.-51. (canceled)

52. The apparatus of claim 37, wherein the first and second SAW generators emit an acoustic power of ranging from 19 to 31 dBm.

53.-121. (canceled)
